(19) United States
(12) Patent Application Publication
(10) Pub. No.: US 2023/0381173 A1
(43) Pub. Date: Nov. 30, 2023

(54) METHODS AND PHARMACEUTICAL COMPOSITION FOR TREATING DISEASES

(71) Applicant: TONGLI BIOMEDICAL CO. LTD., Suzhou (CN)

(72) Inventors: Mingxin QIA, Suzhou, Jiangsu (CN); Rodney HO, Suzhou, Jiangsu (CN); Ty CHEN, Suzhou, Jiangsu (CN)

(21) Appl. No.: 18/231,391

(22) Filed: Aug. 8, 2023

Related U.S. Application Data
(63) Continuation-in-part of application No. PCT/CN2022/075737, filed on Feb. 9, 2022.
(60) Provisional application No. 63/147,260, filed on Feb. 9, 2021.

(51) Int. Cl. A61K 31/4985; A61K 9/00; A61P 33/00; A61K 9/20; A61K 31/4184; A61K 31/15; A61K 31/352; A61K 47/32; A61K 47/38; A61K 47/40; A61K 47/10

(52) U.S. Cl. CPC A61K 31/4985; A61K 9/0056; A61P 33/00; A61K 9/2054; A61K 9/2013; A61K 9/2027; A61K 9/2009; A61K 31/4184; A61K 31/15; A61K 31/352; A61K 47/32; A61K 47/38; A61K 47/40; A61K 47/10

(57) ABSTRACT

A new polymorphic form of (R)-praziquantel is provided, the novel anhydrate (R)-2-(cyclohexanecarbonyl)-2,3,6,7-tetrahydro-1h-pyrazino[2,1-a]isoquinolin-4(11bh)-one, compositions including the polymorphic form, methods of making pharmaceutical formulations including the polymorphic form. Improved methods of their use for controlling, treating and preventing zoonotic and parasitic diseases in humans and animals is also provided, which includes administering to the subject in need thereof a therapeutically effective amount of the active agent with a pharmaceutical formulation.

### CROSS-REFERENCE TO RELATED APPLICATIONS

This application is a continuation-in-part application of PCT Application No. PCT/CN2022/075737, having a filing date of Feb. 9, 2022, which claims priority to U.S. Application No. 63/147,260, having a filing date of Feb. 9, 2021, the entire contents both of which are hereby incorporated by reference.

### FIELD OF TECHNOLOGY

The following relates in general to a new polymorphic form of (R)-praziquantel, anhydrous (R)-2-(cyclohexanecarbonyl)-2,3,6,7-tetrahydro-1h-pyrazino[2,1-a]isoquinolin-4(11bh)-one, compositions comprising the polymorphic form, methods of making pharmaceutical formulations comprising the polymorphic form and methods of their use for the treatment of diseases.

### BACKGROUND

Parasitic flatworms of the phylum Platyhelminthes represent a diverse spectrum of organisms that infect (live within) and infest (live upon) vertebrates. In addition to common parasitic infections and zoonosis in humans, pet animals and production animals, aquaculture including fish serve as both definitive and intermediate hosts for a spectrum of platyhelminth parasites. It is estimated that more than 500 million people suffer or at risk from numerous parasitic zoonosis around the world, which pose a great threat to global health for people and animals.

Praziquantel (PZQ) is a synthetic pyrazine isoquinoline derivative, and a white or off-white crystalline powder and bitter in taste. It is recognized worldwide as a highly effective and broad-spectrum antiparasitic drug against a broad range of cestodes (tapeworms) and trematodes (flukes). Praziquantel is a key anti-platyhelminth parasite therapy in both human and veterinary medicine. It is widely used for treating diseases such as schistosomiasis japonica, schistosomiasis haematobium, schistosomiasis mansoni, clonorchiasis, paragonimiasis, sparganosis mansoni, fasciolopsis, echinococcosis, taeniasis, cysticercosis, etc. In addition to human use, it is also widely used in anti-parasitic treatment of animals including poultry livestock and aquaculture.

Currently marketed product Praziquantel is a racemic compound composed of 50% each of (R)-praziquantel and (S)-praziquantel isomers. Scientists have separated and obtained both pure optic isomers from racemic praziquantel, and found in preclinical studies and preliminary clinical trials that: (R)-praziquantel is the active parasiticidal component of praziquantel, while the (S)-praziquantel is inactive, bitter and even harmful. Therefore, the development of (R)-praziquantel has potential clinical values of higher efficacy, less side effects, and better medical compliance.

It's well known that many small molecule drugs or active pharmaceutical ingredients (APIs) can exist in different crystal forms i.e., polymorphs. In particular, the inclusion of water into the crystal lattice and thus the formation for crystalline hydrates generates challenging drug substance for formulation due to their thermodynamic uncertainty. Variations in hydrate stability in addition to the existence of stoichiometric and non-stoichiometric behavior pose additional problems, which require non-trivial solutions. The polymorphic forms may unpredictably exhibit undesirable physical, chemical, and pharmaceutical characteristics to affect the solubility, stability and bioavailability. This unpredictability of polymorph crystalline form or mixture thereof may lead to the safety and efficacy of these drug products when administered to humans and animals. Therefore, the physicochemical properties, manufacturing, shelf life and biological activity of the drug substance and pharmaceutical products are strictly regulated by the U.S. Food and Drug Administration and other regulatory bodies around the world.

(R)-praziquantel is known in general to exist as hemihydrate or monohydrate forms (Meyer et al., 2009 and Cedillo-Cruz et al., 2014). It is estimated that the theoretical weight loss of this molecule for a mono-hydrate is 5.7% and 2.8% for a hemihydrate. The Chinese invention patent CN104327077A has disclosed a crystal form of (R)-praziquantel, the X-ray diffraction pattern (Cuk α radiation) of which shows the following seven diffraction peaks: 2-Theta 6.9±0.2°, 8.3±0.2°, 15.1±0.2°, 17.4±0.2°, 19.8±0.2°, 21.9±0.2°, and 24.3±0.2°. By far, however, reports of formulations comprising specific (R)-praziquantel crystal form and its storage stability have not been found yet.

As the active pharmaceutical ingredient is often exposed to various pharmaceutical processing and storage conditions, including stress related temperature, solvent and humidity, this creates challenges in controlling in potential transformation of the hydrate and chiral drug molecule during pharmaceutical processing and storage, as well as in development of final formulation. Accordingly, it's desirable to identify and characterize new crystalline forms of (R)-praziquantel that provide advantages relative to other solid-state forms in making, processing, formulating, or administering the compound are desirable. The ability to be able to manufacture the selected polymorphic form reliably is also a critical factor in determining the success of the end drug product.

It has now been surprisingly discovered that (R)-praziquantel can exist as an extremely stable anhydrous crystalline. This unusually stable form demonstrates reproducibly high purity of the drug substance and a desired physical-chemical and pharmaceutical characteristics that can be developed as a drug product on an industrial scale.

### SUMMARY

An aspect relates to a new polymorph of (R)-praziquantel namely Form B.

In one aspect, Form B is a (R)-praziquantel anhydrate and has a characteristic peak in the XRPD pattern at a value of two theta of 13.1±0.2°, 13.1±0.1°. As used herein the term “anhydrate” refers to a crystalline form of a compound which contains not more than 0.2 moles of water per molecule of the compound, so that it can be described as substantially anhydrous.

In a further aspect, Form B is a (R)-praziquantel anhydrate with a water content between 0 and about 1% w/w (0.17 moles of water equivalent) assayed by standard Karl Fischer detection procedure, between about 0 and about 0.6%, between about 0 and about 0.5%. In a detailed aspect, Form B is a (R)-praziquantel anhydrate with a water content of about 0.26%.

In one aspect, the crystalline form B of embodiments of the invention is substantially isolated. By “substantially isolated” is meant that a particular crystalline form of the compound of Formula B is at least partially isolated from impurities. For example, in some embodiments, a crystalline Form B comprises less than about 1%, or less than about 0.5% of impurities. Impurities generally include anything that is not the substantially isolated crystalline form including, for example, other crystalline forms and other substances as well as its optical isomer.

In one aspect, (R)-praziquantel Form B is (R)-2-(cyclohexanecarbonyl)-2,3,6,7-tetrahydro-1H-pyrazino[2,1-a] isoquinolin-4(11bH)-one, with a content of crystal water less than 0.2 mol per molecular.

In certain aspects, (R)-praziquantel Form B is (R)-2-(cyclohexanecarbonyl)-2,3,6,7-tetrahydro-1H-pyrazino[2,1-a] isoquinolin-4(11bH)-one, with a content of crystal water being about 0.1 mol per molecular.

In certain aspects, (R)-praziquantel Form B is (R)-2-(cyclohexanecarbonyl)-2,3,6,7-tetrahydro-1H-pyrazino[2,1-a] isoquinolin-4(11bH)-one, with no crystal water.

In further aspects, the X-ray diffraction pattern (Cuk α radiation) of (R)-praziquantel Form B shows a diffraction peak at 8.6±0.2° with a 100% relative intensity.

In further aspects, the X-ray diffraction pattern (Cuk α radiation) of (R)-praziquantel Form B shows a diffraction peak at 13.1±0.2° with a relative intensity more than 3%. In some embodiments, the relative intensity of the diffraction peak at 13.1±0.2° is lower than 7%. In further embodiments, the relative intensity of the diffraction peak at 13.1±0.2° is lower than 5%.

In further aspects, the X-ray diffraction pattern (Cuk α radiation) of (R)-praziquantel Form B shows a diffraction peak at 17.9±0.2°.

In further aspects, the X-ray diffraction pattern (Cuk α radiation) of (R)-praziquantel Form B shows a diffraction peak at 19.3±0.2°.

In further aspects, the X-ray diffraction pattern (Cuk α radiation) of (R)-praziquantel Form B shows a diffraction peak at 20.1±0.2°.

In further aspects, the X-ray diffraction pattern (Cuk α radiation) of (R)-praziquantel Form B shows a diffraction peak at 20.9±0.2°.

In further aspects, the X-ray diffraction pattern (Cuk α radiation) of (R)-praziquantel Form B shows a diffraction peak at 22.3±0.2°.

In further aspects, the X-ray diffraction pattern (Cuk α radiation) of (R)-praziquantel Form B shows a diffraction peak at 24.8±0.2°.

In further aspects, the X-ray diffraction pattern (Cuk α radiation) of (R)-praziquantel Form B shows a diffraction peak at 27.8±0.2°.

In further aspects, the X-ray diffraction pattern (Cuk α radiation) of (R)-praziquantel Form B shows a diffraction peak at 31.2±0.2°.

In further aspects, the X-ray diffraction pattern (Cuk α radiation) of (R)-praziquantel Form B shows a diffraction peak at 34.5±0.2°.

In some aspects, the X-ray diffraction pattern (Cuk α radiation) of (R)-praziquantel Form B shows diffraction peaks at 7.2±0.2°, 8.6±0.2°, 13.1±0.2°, 15.3±0.2°, 17.9±0.2°, 19.3±0.2°, 20.1±0.2°, 22.3±0.2°, 24.1±0.2°, and 24.8±0.2°.

In another aspects, the X-ray diffraction pattern (Cuk α radiation) of (R)-praziquantel Form B shows diffraction peaks at 7.2±0.2°, 8.6±0.2°, 13.1±0.2°, 13.8±0.2°, 14.4±0.2°, 15.3±0.2°, 16.0±0.2°, 16.9±0.2°, 17.9±0.2°, 18.2±0.2°, 19.3±0.2°, 20.1±0.2°, 20.9±0.2°, 22.3±0.2°, 24.1±0.2°, 24.8±0.2°, 25.8±0.2°, 27.8±0.2°, and 31.2±0.2°.

In another aspects, the X-ray diffraction pattern (Cuk α radiation) of (R)-praziquantel Form B shows diffraction peaks at 7.2±0.2°, 8.6±0.2°, 13.1±0.2°, 15.3±0.2°, 16.0±0.2°, 17.9±0.2°, 18.2±0.2°, 19.3±0.2°, 20.1±0.2°, 20.9±0.2°, 22.3±0.2°, 24.1±0.2°, 24.8±0.2°, 27.8±0.2°, 31.2±0.2°, and 34.5±0.2.

In another aspects, the X-ray diffraction pattern (Cuk α radiation) of (R)-praziquantel Form B shows diffraction peaks at 7.2±0.2°, 8.6±0.2°, 13.1±0.2°, 13.8±0.2°, 14.4±0.2°, 15.3±0.2°, 16.0±0.2°, 16.9±0.2°, 17.9±0.2°, 18.2±0.2°, 19.3±0.2°, 20.1±0.2°, 20.9±0.2°, 22.3±0.2°, 24.1±0.2°, 24.8±0.2°, 25.8±0.2°, 27.8±0.2°, 31.2±0.2° and 34.5±0.2.

In some aspects, the water content of (R)-praziquantel Form B is below 0.6% (w/v). In some other aspects, the water content of (R)-praziquantel Form B is below 0.3%, assayed by standard Karl Fischer detection procedure.

Physically, the anhydrous crystals of one aspect of the present disclosure are obtained as a white powder consisting of aggregates of birefringent rod-like or needle-like crystals.

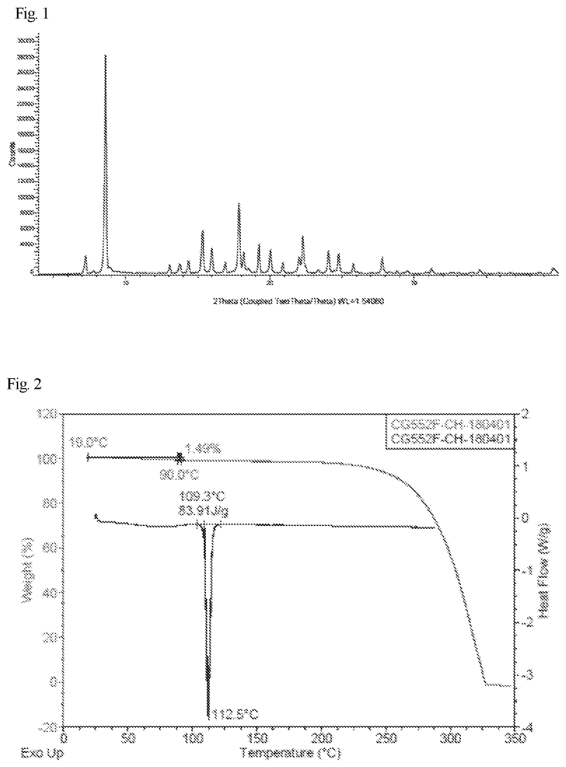

As a typical example, the X-ray diffraction pattern (Cuk α radiation) of (R)-praziquantel Form B is substantially the same with FIG. 1.

As a typical example, the anhydrous crystalline Form B is characterized by a differential scanning calorimetry thermogram with an endotherm having a peak temperature of about 112.5° C.

New crystal form B of (R)-praziquantel has much less water content compared to all known hemihydrate and monohydrate crystalline forms. Surprisingly, it also demonstrates a long-term stability during various storage condition.

One aspect of the present disclosure provides pharmaceutical formulations comprising one or more crystalline forms of (R)-praziquantel especially Form B capable of advantageous therapeutic actions. It is a related object to provide compositions and the methods to make the pharmaceutical formulations of (R)-praziquantel capable of eliciting and sustaining desirable pharmacokinetic responses in the subject in need thereof. The crystals of embodiments of the present invention can be administered alone or in the form of a pharmaceutical composition in combination with pharmaceutically acceptable carriers or excipients, the proportion and nature of which are determined by the solubility and chemical properties of the compound selected, the chosen route of administration, and standard pharmaceutical practice. It is another object of this disclosure to provide methods for treating schistosomiasis and other disorders mediated by infections caused by trematodes, taeniasis and other parasites using such formulations. In an embodiment, the subject may be an animal, warm-blooded animal, mammal, e.g., human or non-human mammal or fish.

For oral administration, the compositions may be formulated as tablets, capsules, pills, granules, chews, dragees, troches, liquids, semi-solid gels and pastes, syrups, slurries, suspensions and the like, for oral ingestion by a subject to be treated.

Also for intravenous administration or subcutaneous administration, the compositions may be formulated in solutions, in physiologically compatible buffers such as Hanks's solution, Ringer's solution, or physiological saline buffer. The solution may contain formulary agents such as suspending, stabilizing and/or dispersing agents. In a particular embodiment, the compositions are formulated in sterile solutions.

The compositions can further comprise a pharmaceutically acceptable carrier. The term “carrier” refers to a diluent, excipient, or vehicle with which the therapeutic is administered. The carrier or excipient may be a solid, semi-solid, or liquid material, which can serve as a vehicle or medium for the active ingredient. In addition to the formulations described previously, the composition may also be formulated as a depot preparation. Such formulations may be administered by implantation (for example subcutaneously or intramuscularly) or by intravascular or intramuscular injection.

Additionally, the composition may be delivered using a sustained-release system, such as semi-permeable matrices of solid polymers containing the composition.

For oral administration, the pharmaceutical compositions take the form of, for example, tablets or capsules or granules or chews or pastes prepared by conventional means with pharmaceutically acceptable excipients such as binders (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid or semi-solid preparations for oral administration may take the form of, for example, solutions, syrups or suspensions, or they may be presented as a soft chew or paste product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); solubilizers (e.g. cyclodextrins), non-aqueous vehicles (e.g., hydrogenated vegetable oils, almond oil, oily esters or ethyl alcohol); and preservatives (e.g., methyl or propyl p-hydroxybenzoates or sorbic acid, benzyl alcohol, sodium benzoate, potassium sorbate).

In accordance with this disclosure, there is provided, in one embodiment, a tablet having a pharmaceutical formulation of (R)-praziquantel Form B according to present disclosure.

In further embodiments, the pharmaceutical formulation includes (R)-praziquantel Form B and pharmaceutically acceptable excipients. In one embodiment, the pharmaceutical formulation includes 40-80% of (R)-praziquantel Form B by weight and 20-60% of pharmaceutically acceptable excipients by weight. In another, the pharmaceutical formulation includes 50-70% of (R)-praziquantel Form B by weight and 30-50% of pharmaceutically acceptable excipients by weight.

Further, the excipients include disintegrants, binders, fillers, surfactant/solubilizers, glidants, flavoring agents, diluents, anti-adherents, lubricants, coatings etc.

Examples of disintegrants include one or more, but not limited to hydroxypropyl cellulose (HPC), low density HPC, carboxymethyl starch sodium, carboxymethylcellulose (CMC), sodium CMC, calcium CMC, carboxymethyl starch, hydroxypropyl starch, modified starch; crystalline cellulose, sodium starch glycolate (CMS-Na); calcium carbonate, croscarmellose sodium, crospovidone, gums, magnesium aluminum silicate, methylcellulose, polacrilin potassium, sodium alginate, low substituted hydroxypropyl cellulose, and cross-linked polyvinylpyrrolidone hydroxypropyl cellulose, sodium starch glycolate, and starch.

In an embodiment, the disintegrants may be present in an amount ranging from about 2% to about 30% by weight of the formulation. In an embodiment, the disintegrants may be present in an amount ranging from about 2% to about 10% by weight of the formulation.

In one embodiment, the disintegrant is sodium starch glycolate (CMS-Na), and its content is 2-8%.

Examples of binders include one or more, but not limited to polyvinyl pyrrolidone (also known as povidone), povidone K30, polyethylene glycol(s), acacia, alginic acid, agar, calcium carrageenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or mixtures thereof. In an embodiment, the binders may be present in an amount ranging from about 1% to about 20% by weight of the formulation.

In one embodiment, the binder is povidone K30, and its content is 2-8%.

Examples of diluents or fillers include one or more, but not limited to lactose (for example, spray-dried lactose, a-lactose, β-lactose) available under the trade mark Tablettose, various grades of lactose available under the trade mark Pharmatose or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose (MCC) available under the trade mark Avicel), microcrystalline cellulose PH101, hydroxypropyl cellulose, L-hydroxypropyl cellulose (low substituted), hydroxypropyl methylcellulose (HPMC), methylcellulose polymers (such as, for example, Methocel A, Methocel A4C, Methocel A15C, Methocel A4M), hydroxy ethylcellulose, sodium carboxymethyl cellulose, carboxymethylene, carboxymethyl hydroxyethyl cellulose, Non-Pareil Seeds, sugar spheres and other cellulose derivatives, soy protein fines, starches or muddied starches (including potato starch, corn starch, maize starch and rice starch) and mixtures thereof. In an embodiment, the carriers, diluents or fillers may be present in an amount ranging from about 2% to about 50% by weight of the composition.

In one embodiment, the diluent or filler is MCC, and its content is 35-45%.

Examples of surfactants or solubilizers comprise one or more selected from the group consisting of one or more, but not limited to polysorbates (tween 80), sodium dodecyl sulfate (sodium lauryl sulfate, SLS), lauryl dimethyl amine oxide, docusate sodium, poloxamers, (poloxamer 188) cetyl trimethyl ammonium bromide (CTAB), polyethoxylated alcohols, polyoxyethylene sorbitan monooleate, octoxynol, N, N-dimethyldodecylamine-N-oxide, hexadecyltrimethylammonium bromide, polyoxyethylene 10 lauryl ether, brij, bile salts (sodium deoxycholate, sodium cholate), polyoxyl castor oil, 2-hydroxylpropyl-beta-cyclodextrin (HPβCD), β-cyclodextrin, sulfobutylether β-cyclodextrin (SBEβCD), lecithin, methylbenzethonium chloride. carboxylates, sulphonates, petroleum sulphonates, alkylbenzene sulphonates, naphthalene sulphonates, olefin sui phonates, alkyl sulphates, sulphates, sulphated natural oils & fats, sulphated esters, sulphated alkanolamides, alkylphenols, ethoxylated & sulphated, ethoxylated aliphatic alcohol, polyoxyethylene surfactants, carboxylic esters polyethylene glycol esters, anhydro sorbitol ester & it's ethoxylated derivatives, glycol esters of fatty acids, carboxylic amides, monoalkanolamine condensates, polyoxyethylene fatty acid amides, quaternary ammonium salts, amines with amide linkages, polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethyl glycine sodium salt or mixtures thereof.

In an embodiment, the surfactants or solubilizer may be present in an amount ranging from about 0.5% to about 10% by weight of the formulation.

In one embodiment, the surfactant or solubilizer is SLS, and its content is 1-3%. In an embodiment, the content of SLS in the tablet is 0.9-1%.

In an embodiment, glidants, anti-adherents and lubricants are incorporated in the pharmaceutical formulations of the present disclosure, which may comprise one or more, but not limited to stearic acid and pharmaceutically acceptable salts or esters thereof (for example, magnesium stearate, calcium stearate, sodium stearyl fumarate or other metallic stearate), talc, waxes (for example, microcrystalline waxes) and glycerides, light mineral oil, Polyethylene glycol (PEG), silica acid or a derivative or salt thereof (for example, silicates, silicon dioxide, colloidal silicon dioxide and polymers thereof, crospovidone, magnesium aluminosilicate and/or magnesium aluminometasilicate, sucrose ester of fatty acids, hydrogenated vegetable oils (for example, hydrogenated castor oil), or mixtures thereof. In an embodiment, the glidants, anti-adherents and lubricants may be present in an amount ranging from about 0.1% to about 6% by weight of the formulation. In an embodiment, the glidants, anti-adherents and lubricants may be present in an amount ranging from about 0.1% to about 5% by weight of the formulation or a proper mixture thereof.

In one embodiment, the glidant is colloidal silicon dioxide, and its content is 0.3-2%.

In one embodiment, the lubricant is magnesium stearate, and its content is 0.25-5%.

In one embodiment, the lubricant is sodium stearyl fumarate, and its content is 0.3-3%. Tests shown that with sodium stearyl fumarate as the lubricant, no color change was observed, whereas some kinds of lubricants (e.g. Magnesium stearate) may have problem of color changing when stored under high temperature (eg. 60° C.).

According to yet another embodiment, the pharmaceutical formulation is a tablet, by weight 2-10% of which is disintegrant, 2-30% is binder, 2-40% is filler, 0.5-2% is surfactant (or stabilizer/solubilizer) and 0.3-0.8% is lubricant.

In another embodiment, by weight 2-10% of the tablet is disintegrant, 2-10% is binder, 30-40% is filler, and 0.3-3% is lubricant.

According to still another embodiment, the excipients include povidone K30. In a further embodiment, by weight 1-4% of the tablet is povidone K30.

According to another embodiment, one tablet includes 10-600 mg (R)-praziquantel Form B, 50-600 mg, 20-300 mg, further preferably 50-300 mg, 50-150 mg.

In one embodiment, the tablet includes by weight: 40-80% (R)-praziquantel Form B, MCC 30-50%, SLS 0.5-2%, CMS-Na 2-8%, povidone K30 1-8%, colloidal silicon dioxide 0.3-2%, sodium stearyl fumarate 0.1-5%.

In another embodiment, the tablet includes by weight: 40-60% (R)-praziquantel Form B, MCC 30-50%, SLS 0.5-2%, CMS-Na 5-8%, povidone K30 1-5%, colloidal silicon dioxide 0.3-1%, sodium stearyl fumarate 0.3-3%.

In another embodiment, the tablet includes by weight: 40-60% (R)-praziquantel Form B, MCC 30-50%, SLS 0.5-2%, CMS-Na 5-8%, povidone K30 1-5%, colloidal silicon dioxide 0.3-1%, sodium stearyl fumarate 2-5%.

Further, particle size of (R)-praziquantel Form B is below 150 microns, at least 90% of the particles have a particle size of less than about 100 microns, less than about 50 microns, less than about 20 microns. In another embodiment, at least 90% of the particles have a particle size of less than about 0.2 microns.

In addition, embodiments of the present invention also provide for a semi-solid oral homogeneous anthelmintic veterinary formulation, for the treating, controlling, and preventing of endo- and ectoparasite infections in warm blooded animals, in particular in companion animals such as cat and dogs. This soft oral compositions or pasta comprising essentially of (R)-praziquantel form B and at least benzimidazoles (albendazole) or one of macrocyclic lactones (moxidectin and ivermectin) or tribendimidine or milbemycin oxime or other antiparasitic drugs, may contain further veterinary acceptable inert ingredients, for example, thickeners, humectants, preservatives, pH adjusting agents, colorants, flavors, binders, fillers, viscosity modifier and an opacifier or dispersing agents, antioxidants and buffers.

The solvents provided for in the inventive homogeneous pastes are those polar solvent that will dissolve both the first anthelmintic agent (R)-praziquantel and at least one benzimidazoles (albendazole) or tribendimidine, or a macrolide anthelmintic compound (milbemycin oxime). These solvents include, for example, propylene glycol, polyethylene glycol, glycerol formal, 1-methylpyrrolidone (NMP), dimethyl sulfoxide (DMSO).

The thickeners contemplated by embodiments of the invention are well known to a practitioner of this art. Compounds which function as thickeners include, for example, celluloses, starches, natural gums, monothioglycerol, Synthetic polymers, such as polymers and copolymers of polyvinylpyrrolidone or (meth)acrylates, etc. Especially desired thickeners are hydroxypropyl cellulose, xanthan gum and hydroxyethyl starch. Thickeners may be present in amounts of from about 3% to about 30%.

Opacifiers may be added to absorb and/or reflect certain light and/or energy of certain wavelengths and may thus enhance the stability of the formulations. Opacifiers include, for example, zinc oxide or titanium dioxide and may be present in amounts from about 0.5 to 2.5%. Titanium dioxide is especially desired. These compounds are well known to practitioners of this art.

Humectants that may be used in the compositions include, but are not limited to, glycerol (also referred to herein as glycerin), propylene glycol, cetyl alcohol and glycerol monostearate, and the like. Polyethylene glycols of various grades may also be used as humectants.

Many flavoring agents may be used in the formulations of the disclosure to improve the palatability of the oral veterinary formulations. Flavoring agents are those that are not derived from animal sources. In various embodiments, flavoring components derived from fruit, meat (including, but not limited to pork, beef, chicken, fish, poultry, and the like), vegetable, cheese, bacon, cheese-bacon and/or artificial flavorings may be used.

The inventive formulations may contain other inert ingredients such as antioxidants. These compounds are well known in the formulation art. Antioxidants may be added to the compositions of embodiments of the invention to inhibit degradation of the active agents. Suitable antioxidants include, but are not limited to, ascorbic acid, fumaric acid, sodium ascorbate, BHA (butylated hydroxy anisole) and the like. The antioxidants are generally added to the formulation in amounts of from about 0.01 to about 2.0% (w/w), based upon total weight of the formulation, with about 0.05 to about 1.0% especially desired.

In accordance with this disclosure, there is provided, in another embodiment, a method for treating a parasitic disease in a human or an animal. In embodiments, the method comprises administering the aforementioned formulation to a subject suffering from the parasitic disease.

Further, the pharmaceutical formulation containing (R)-praziquantel shows good prospect in treating many zoonotic and parasitic diseases, including, but not limited to, schistosomiasis, clonorchiasis, paragonimiasis, or opisthorchiasis, fasciolopsiasis, taeniasis, cysticercosis and hydatid disease, In particular, the pharmaceutical formulation may be used for treating infections caused by the species of Schistosoma, e.g., Schistosoma mekcongi, Schistosoma japonicum, Schistosoma mansoni and Schistosoma haematobium, and infections due to the liver flukes, Clonorchis sinensis, Opisthorchis viverrini or the gastrointestinal infection caused by tapeworms, including Dipylidium caninum or Taenia taeniaeformis. The pharmaceutical formulation may be used for the treatment of cysticercosis and neurocysticercosis (NCC). The pharmaceutical formulation is also useful for the treatment of health conditions that may be treated combined with other anthelmintics, antischistosomals, and antitrematodes. Further, the pharmaceutical formulation may be used in veterinary medicine, for example, in dogs and cats for the removal of tapeworm, and for the removal of various types of hookworms, roundworms, whipworms and filaria such as Dirofilaria immitis in combination with benzimidazoles or tribendimidine or microllide compounds (moxidectin, selamectin, milbemycin oxime) and cyclo-octadepsipeptides (Emodepside). The pharmaceutical formulation may eliminate tapeworms in humans, buffalo, sheep, swine, horses, ferrets, birds, chinchillas, mice, rats, hamsters, gerbils, and guinea pigs, still other, the pharmaceutical formulation may be used in aquaculture and fish for removing tapeworms and flukes in various fishes and reptiles.

In some embodiments, the parasite is a tapeworm, which is a flat, segmented worm that lives in the intestines of mammals. During infection, live tapeworm larvae are grouped in cysts. Once inside the digestive tract, a larva can grow into a large adult tapeworm, which causes symptoms in the host. Tapeworms can cause gastrointestinal infections. For example, cysticercosis is one disease involving larval tapeworms in the human body. The tapeworm comprises Taenia solium, Taenia saginata, Hymenolepis nana, Dipylidium caninum, and Taenia laeniaeformis.

In some embodiments, the parasite is Monogenean in aquatic animals, including but not limited to, Ancylodiscoides vistulensis, Benedenia seriolae, Neobenedenia, Cleidodiscus sp., Clemacotyle australis, Dactylogyrus sp., Gyrodactylus aculeati, Gyrodactylus turnbulli, Haliotrema abaddon, Lepidotrema bidyanain in European catfish, Yellowtail amberjack, Black crappie, Whitespotted eagle rays, goldfish, guppy, stickleback, West Australian dhufish, silver perch, golden pomfrets, yellow croaker, eels, and Digenean, Clinostomum complanatum, Clinostomum marginatum, Diplostomum spathaceum insunshine bass, channel catfish, grass carp, silver carp, and Cestode (Bothriocephalus acheilognathi) in Bonytail chub, grass carp, red shiner, red snapper, as well as blood flukes in bluefin tuna, Cardicola forsteri, Thunnus maccoyii, Thunnus thynnus, Thunnus orientalis and Cardicola orientalis.

According to some embodiments, the additional suitable anti-parasitic medications that can combine with the (R)-praziquantel crystal form B for human and animal use is one or more selected from the group consisting of tinidazole, metronidazole, melarsoprol, eflornithine, rifampin, amphotericin B, pentamidine, sodium stibogluconate, meglumine antimoniate, fluconazole, artesunate, artemether, and dihydroartemisinin, quinine, quinidine, chloroquine, atovaquone-proguanil, artemether-lumefantrine, mefloquine, doxycycline, clindamycin, paromomycin, atovaquone, nitazoxanide, azithromycin, fumagillin, paromomycin, diloxanide, levamisole, secnidazole, ornidazole, iodoquinol, diloxanide furoate, clindamycin, atovaquone, azithromycin, diminazen, trypan blue, oxamniquinine, niclosamide, emodepside, albendazole, fenbendazole, mebendazole, tribendimidine, thiabendazole, pyrantel, fenbentel, morantel, monepantel, derquantel, diethylcarbamazine, milbemycin Oxime, ivermectin, eprinomectin, selamectin, doramectin, moxidectin, and abamectin.

In accordance with this disclosure, there is provided, in yet another embodiment, a method for modulating response of profibrotic and pro-inflammatory cytokines and chemokines in a subject suffering from a disorder mediated by such cytokines during the course of chronic schistosomiasis or other diseases. In embodiments, the method comprises administering the aforementioned formulations to the subject. Examples of such cytokines include integrin, fibronectin, interferon-γ, interleukin (IL)-2, IL-4, IL-5, IL-6, IL-10, TNF-α and TGF-β1 produced by host immune cells. Examples of such disorder include inflammation and autoimmune, regeneration and hepatic fibrotic diseases caused by chronic schistosomiasis. In embodiments, the method comprises administering the (R)-praziquantel crystal form B formulations to the subject.

The treatment contains the (R)-praziquantel crystal are effective over a wide dosage range. Typical dosage forms comprise the (R)-praziquantel crystal form provided herein, thereof lie within the range of from about 0.1 mg to about 4500 mg per day, given as a single once-a-day dose in the morning or as divided doses throughout the day taken with food. Dosage unit can have about 0.1, 0.2, 0.3, 0.4, 0.5, 1.0, 2.0, 2.5, 5.0, 10.0, 15.0, 20.0, 25.0, 50.0, 100, 200, 250, 300, 350, 400, 450, 500, or 1000 mg of the active compound.

In still another embodiment, the method further comprises administering one or more formulations to the subject one or more times a day, with a total daily intake of (R)-praziquantel Form B greater than 10 mg/kg for a human and 1 mg/kg for a dog or cat. In some embodiments, the formulation is a tablet. In other embodiments, the formulation is a capsule, a chew or a paste.

In various embodiments, the attending physician may change the doses, dosing route and dosing regimen in view of the age, health and sex of the subject as well as the severity of the disease, and therefore the above dosage ranges are not intended to limit the scope of embodiments of the invention in any way. The pharmaceutical compositions will be typically administered singly or divided into several doses daily or consecutive days.

In accordance with this disclosure, there is provided, in still another embodiment, a tablet and other formulation compositions having an effective amount of (R)-praziquantel Form B and pharmaceutically acceptable excipients. For example, the tablet when administered in a subject can sustain a measurable pharmacokinetic response. The pharmacokinetic response is characterized by a shorten T max and an increased in plasma exposure (AUC and Cmax values) for the (R)-praziquantel tablet compared to those for an equivalent dose of R-PZQ oral dispersible tablet or a capsule or a marketed racemic praziquantel tablet containing 50% of R-PZQ as active isomer while another 50% (S)-PZQ as an inactive, bitter isomer (see Example 8).

In various embodiments, treatment methods of administering such tablets and other formulation compositions are provided for subjects (patients or animals) suffering from zoonotic parasitic diseases such as schistosomiasis and liver, lung fluke infections and neurocysticercosis, taeniasis, hydatidosis, and other autoimmune and inflammatory fibrotic disorders mediated by worm-specific cytokine in the course of the infections.

In various embodiments, treatment methods of administering such active pharmaceutical ingredient are provided for subjects (patients or animals) suffering from zoonotic parasitic diseases in warm blood animals and in particular, cats, dogs, horses, chickens, pigs, sheep and cattle as well as birds.

In various embodiments, treatment methods of administering such active pharmaceutical ingredient are provided by bath treatment for aquaculture including production fish and ornamental fish suffering from zoonotic parasitic diseases and other diseases such as skin diseases infected with monogenean parasites.

In various embodiments, treatment methods of administering such active pharmaceutical ingredient are provided by oral intubation or medicated fish feed for aquaculture including production fish and ornamental fish as a definitive/intermediate host, including but not limited to, European catfish, Yellowtail amberjack, Cownose rays, Black Crappie, White-spotted eagle, rays, Goldfish, Guppy Stickleback, Rainbow trout, West Australian dhufish, Takifugu rubripes, Silver perch, Rockfish, Chub mackerel, Pacific Bluefin Tuna, golden pomfrets, yellow croaker, eel, Sunshine bass, Channel, catfish, Grass carp, Silver carp, Bluegill, Common carp, Bonytail chub, Red Shiner, Red snapper, Turbot.

In one aspect, the present disclosure refers to a preparation method of (R)-2-(cyclohexanecarbonyl)-2,3,6,7-tetrahydro-1H-pyrazino[2,1-a] isoquinolin-4(11bH)-one crystal Form B, which comprises:
a) Dissolving the compound in at least one solvent at around 45° C. to form a solution;
b) Adding another solvent to the solution and cooling down to form a solid;
c) Isolating the solid by filtration;
d) Drying the solids under vacuum (≤−0.08 MPa) at 35±5° C. for at least 10 hours to get the Form B.

In an embodiment, the one solvent is an anhydrous solvent. In an embodiment, the one solvent is selected from acetone, acetonitrile, chloroform, dichloromethane, dimethyl sulfoxide, 1,2-dimethoxyethane, 1,4-dioxane, ethanol, ethyl acetate, isopropyl acetate, isopropyl alcohol, methanol, methyl isobutyl ketone, 2-methyltetraohydrofuran, methyl tert-butyl ether, N,N-dimethylacetamide, N-methyl pyrrolidone, tetrahydrofuran, toluene, and the antisolvent is selected from n-heptane, n-hexane, petroleum ether and water, etc., or a combination thereof.

In an embodiment, in step b), cooling down the mixture at 0˜5° C., preferable at 3° C. for at least 5 hours.

### BRIEF DESCRIPTION

Some of the embodiments will be described in detail, with references to the following Figures, wherein like designations denote like members, wherein:

FIG. 1 provides a representative X-ray powder diffraction (XRPD) pattern of (R)-praziquantel Form B. In this figure the counts per 2 seconds (linear scale) are presented on the y-axis, while the 2 theta values in degrees are presented on the X-axis.

FIG. 2 provides a representative thermogravimetric analysis (TGA) curve and a representative differential scanning calorimeter (DSC) thermogram of Form B. The temperature in ° C. is shown on the X-axis. The heat flow in W/g is shown on the right-hand ordinate (lower curve), while the weight loss in % is shown on the left-hand ordinate (upper curve).

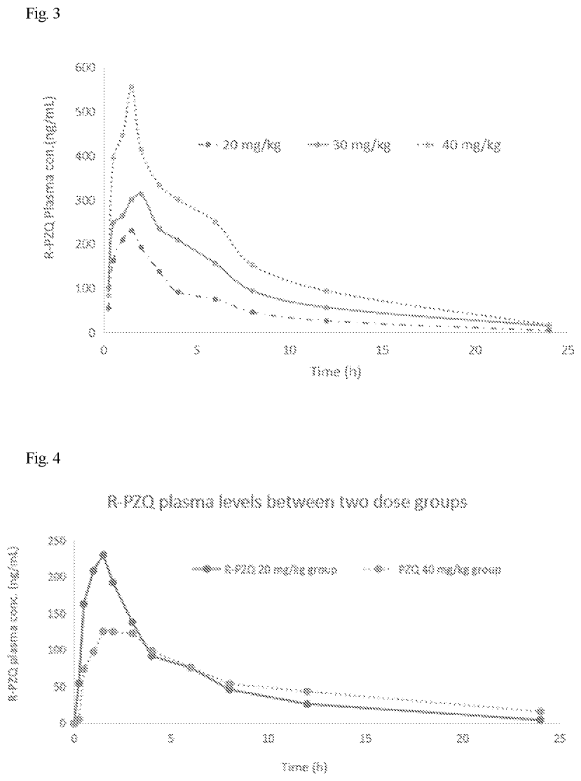

FIG. 3 shows average (R)-praziquantel plasma concentrations over time profiles in healthy human subjects after a single dose of 20 mg/kg, 30 mg/kg and 40 mg/kg TL-010 dosed orally in tablets, respectively, according to Example 8 in this disclosure.

FIG. 4 shows average (R)-praziquantel plasma concentrations over time profiles in health human subjects in a cross-over study design after receiving a single dose of 20 mg/kg TL-010 tablet and 40 mg/kg reference drug racemic praziquantel tablet (Biltricide®), respectively, according to Example 8 in this disclosure.

### DETAILED DESCRIPTION

Hereinafter, various embodiments of this document will be described. Embodiments and terms used herein are not intended to limit the technologies described in the present disclosure to specific embodiments, and the embodiments and the terms include modifications, equivalents, and/or alternatives on the corresponding embodiments described herein.

### Discussion of the Relevant Terms

Throughout the present disclosure relevant terms are to be understood consistently with their typical meanings established in the relevant art, i.e., the art of pharmaceutical chemistry, medicine, biology, parasitology, genetics, molecular biology, biochemistry, physiology and pharmacology. However, further clarifications and descriptions are provided for certain terms as set forth below:

The words “comprise”, “comprising”, “include”, “including” and “includes” when used in this specification and claims are intended to specify the presence of stated features, integers, components, or steps, but they do not preclude the presence or addition of one or more other features, integers, components, steps, or groups thereof.

As used herein, the term “about” when used about X-ray powder diffraction pattern peak positions refers to the inherent variability of the peaks depending on, for example, the calibration of the equipment used, the process used to produce the polymorph, the age of the crystallized material and the like, depending on the instrumentation used. In this case the measure variability of the instrument was about 0.2 degrees 2-theta (θ). A person skilled in the art, having the benefit of this disclosure, would understand the use of “about” in this context. The term “about” in reference to other defined parameters, e.g., water content, C, t, AUC, intrinsic dissolution rates, temperature, and time, indicates the inherent variability in, for example, measuring the parameter or achieving the parameter. A person skilled in the art, having the benefit of this disclosure, would understand the variability of a parameter as connoted by the use of the word “about”.

The term “chiral” refers to molecules which have the property of non-superimposability of the mirror image partner. “Enantiomers” refer to two stereoisomers of a compound which are non-Superimposable mirror images of one another. Many organic compounds exist in optically active forms, i.e., they have the ability to rotate the plane of plane-polarized light. In describing an optically active compound, the prefixes L and D, or R and S. are used to denote the absolute configuration of the molecule about its chiral center(s). The prefixes L and D, or R and S or (−) and (+) are employed to designate the sign of rotation of plane-polarized light by the compound, with L or R or (−) meaning that the compound is levorotatory. A compound prefixed with D or S or (+) is dextrorotatory. For a given chemical structure, these stereoisomers are identical except that they are mirror images of one another. A specific stereoisomer may also be referred to as an enantiomer, and a mixture of such isomers is often called an enantiomeric mixture in general. A 50:50 mixture of enantiomers is referred to as a racemic mixture or a racemate, which may occur where there has been no stereo-selection or stereospecificity in a chemical reaction or process.

“Polymorph”, as used herein, refers to the occurrence of different crystalline forms of a compound. Crystalline forms have different arrangements and/or conformations of the molecule in the crystal lattice. Solvates are crystal forms containing either stoichiometric or nonstoichiometric amounts of a solvent. The term “solvate” refers to forms of a compound that are associated with a solvent, usually by a solvolysis reaction. This physical association may include hydrogen bonding. Conventional solvents include water, methanol, ethanol, acetic acid, DMSO, THF, diethyl ether and the like. Therefore, a single compound may give rise to a variety of polymorphic forms where each form has different and distinct physical properties, such as solubility profiles, melting point temperatures, hygroscopicity, particle shape, density, flowability, compactability and/or X-ray diffraction peaks. The solubility of each polymorph may vary, thus, identifying the existence of pharmaceutical polymorphs is essential for providing pharmaceuticals with predictable solubility profiles. It is desirable to investigate all solid-state forms of a drug, including all polymorphic forms, and to determine the stability, dissolution and flow properties of each polymorphic form. Polymorphic forms of a compound can be distinguished in a laboratory by X-ray diffraction spectroscopy and by other methods.

The acronym “XRPD” means X-ray powder diffraction, an analytical technique which measures the diffraction of X-rays in the presence of a solid component. Materials which are crystalline and have regular repeating arrays of atoms generate a distinctive powder pattern. Materials with similar unit cells will give powder patterns that are similar in position as measured in 2θ (theta). Solvates which exhibit this property are called isostructural or isomorphous solvates. The intensity of the reflections varies according to the electron density causing diffraction as well as sample, sample preparation, and instrument parameters. Analysis of XRPD data is based upon the general appearance of the measured powder pattern(s) with respect to the known response of the X-ray diffraction system used to collect the data. For diffraction peaks that may be present in the powder pattern, their positions, shapes, widths and relative intensity distributions can be used to characterize the type of solid-state order in the powder sample. The position, shape and intensity of any broad diffuse scatter (halos) on top of the instrumental background can be used to characterize the level and type of solid-state disorder.

XRPD was performed with a Bruker D2 phaser diffractometer. Approximately 10 mg of the compound was distributed evenly onto a single crystal silicon plate. The instrument parameters are listed in table 1 below:

TABLE 1

| | |
|---|---|
| Step Size[°2Th]: 0.0263 | Scan Step Time [s]: 46.665 |
| K-Alpha1[A]: 1.54060 | K-Alph2[A]: 1.54443 |
| Generator Settings: 40 mA, 45 KV | Scan Range[°2Th]: 3-40 |

Differential scanning calorimetry (DSC) measures the difference in heat energy between a solid sample and an appropriate reference with an increase in temperature. DSC thermograms are characterized by endotherms (indicating energy uptake) and by exotherms (indicating energy release), typically as the sample is heated. The DSC thermographs were obtained using a TA-Instruments TA Discovery 2500. Samples were weighed (about 3 mg) into standard aluminum TA-Instrument sample pan closed with an appropriate cover. Following parameters were used initial temperature: 25° C.; heating rate: 10° C./min: final temperature: 300° C., nitrogen flow: 50 ml/min. For any given example, the observed endotherms may also differ from instrument to instrument; however, it will generally be within the ranges defined herein provided the instruments are calibrated similarly.

Thermal gravimetric analysis (TGA) is a testing procedure in which changes in weight of a specimen are recorded as the specimen is heated in air or in a controlled atmosphere such as nitrogen. Thermogravimetric curves (thermograms) provide information regarding solvent and water content and the thermal stability of materials. TGAs were performed on TA Discovery TGA 5500 thermogravimeter, using the following parameters-initial temperature: room temperature; heating rate: 10° C./min: final condition: 350° C.

A “subject” to which administration is contemplated includes, but is not limited to, humans (i.e., a male or female of any age group, e.g., a pediatric subject (adolescent) or adult subject (e.g., young adult, middle-aged adult, or senior adult) and/or other non-human animals, for example, mammals (commercially relevant mammals such as cats, and/or dogs, horses, cattle, buffalo, pigs, sheep, goats) and birds (e.g., commercially relevant birds such as chickens, ducks, geese, and/or turkeys). In certain embodiments, the animal is a mammal. The animal may be a male or female at any stage of development. The animal may be a transgenic animal or genetically engineered animal. In certain embodiments, the subject is a non-human animal. In certain embodiments, the animal is fish.

The “parasitic disease” refers to an infection or disease caused or transmitted by a parasite. Examples or parasitic diseases include schistosomiasis, clonorchiasis, opisthorchiasis, paragonimiasis and cestodiasis, fasciolopsiasis, lymphatic filariasis, onchocerciasis, dracunculiasis, trichuriasis, stronglyoidiasis, trichostrongyliasis, trichomoniasis, toxoplasmosis, malaria, african trypanosomiasis, chagas disease, leishmaniasis, amebiasis and giardiasis. in another embodiment, the parasitic disease is a human or animal parasite disease, which includes, but is not limited to, tapeworm—tapeworm infection, diphyllobothriasis—tapeworm, echinococcosis—tapeworm, hymenolepiasis, beef tapeworm, cysticercosis-pork tapeworm, bertielliasis, sparganosis, clonorchiasis, lancet liver fluke, liver fluke—fasciolosis, fasciolopsiasis—intestinal fluke, metagonimiasis—intestinal fluke, metorchiasis, chinese liver fluke, paragonimiasis, lung fluke, schistosomiasis—bilharzia, bilharziosis or snail fever (all types), intestinal schistosomiasis, urinary schistosomiasis, schistosomiasis by Schistosoma japonicum, asian intestinal schistosomiasis, swimmer's itch, ancyiostomiasis/hookworm, angiostrongyliasis, anisakiasis, roundworm—parasitic pneumonia, roundworm-baylisascariasis, roundworm-lymphatic filariasis, dioctophyme renaleinfection, guinea worm—dracunculiasis, pinworm-enterobiasis, gnathostomiasis, halicephalobiasis, loa loa filariasis, calabar swellings, mansonelliasis, filariasis, river blindness, onchocerciasis, strongyloidiasis-parasitic pneumonia, thelaziasis, toxocariasis, trichinosis, whipworm, elephantiasis—lymphatic filariasis, acanthocephaliasis, halzoun syndrome, myiasis, screwworm, cochliomyia, chigoe flea, bedbug, human botfly, head louse-pediculosis, body louse—pediculosis, crab louse-pediculosis, demodex-demodicosis, scabies, “chiggers” (trombiculidaev-trombiculosis, flea, siphonaptera, tick, granulomatous amoebic encephalitis (eye infection), acanthamoeba keratitis, granulomatous amoebic encephalitis (skin infection), babesiosis, balantidiasis, blastocystosis, cryptosporidiosis, cyclosporiasis, dientamoebiasis, amoebiasis, giardiasis, isosporiasis, leishmaniasis, primary amoebic meningoencephalitis (PAM), malaria, rhinosporidiosis, sarcocystosis, toxoplasmosis (acute and latent), trichomoniasis, sleeping sickness, and chagas disease.

The terms pharmaceuticals, pharmaceutical products, drug products, drug chemicals, drug compounds, compounds, and chemicals, are used interchangeably throughout this disclosure. API, as used herein, refers to active pharmaceutical ingredients. In various embodiments of this disclosure, when not specified, the API of the capsule or tablet or other formulations is (R)-praziquantel Form B, also named as TL-010. The terms pharmaceutically acceptable excipients, pharmaceutically compatible excipients, and excipients are used interchangeably in this disclosure. They refer to non-API substances such as disintegrants, binders, fillers, and lubricants, solubilizers, used in formulating pharmaceutical products. They are generally safe for administering to humans and animals according to established regulatory standards, including those promulgated by the United States Food and Drug Administration.

Tablet, as used herein, refers to a generally safe, readily dissolvable enclosure for carrying certain pharmaceutical products.

Capsule, as used herein, refers to a generally safe, readily dissolvable enclosure for carrying certain pharmaceutical products. In one embodiment, capsule is made of gelatin. The capsule size is 1-4 in general.

Other suitable matrix substances such as total synthetic polymer chemicals having gelatin-like properties may be used to manufacture TL-010 tablets or capsules or liquid or semi-solid formulations such as soft chews or pastes according to alternative embodiments of this disclosure.

AUC, as used herein, refers to the area under the curve that represents changes in blood concentrations of TL-010 over time.

Cₘₐₓ, as used herein, refers to the maximum value of blood concentration shown on the curve that represents changes in blood concentrations of TL-010 over time.

Tₘₐₓ, as used herein, refers to the time at maximum value of blood concentration shown on the curve that represents changes in blood concentrations of TL-010 over time.

T_{1/2} as used in this disclosure, refers to the time that it takes for TL-010 blood concentration to decline to one-half of the maximum level.

Collectively AUC, Cₘₐₓ, Tₘₐₓ and T_{1/2} are the principle pharmacokinetic parameters that characterize the pharmacokinetic responses of a particular drug product such as TL-010 in an animal or human subject.

Tablet Formulation of (R)-Praziquantel for Human and Animal Use

To those skilled in the pharmaceutical research and manufacturing, it is generally known that tablet formulations permit generous additions of non-API ingredients including excipients and coating substances, especially high percentage of fillers. However, the addition of non-API ingredients may limit the amount of API carried in each tablet.

The present disclosure provides a new (R)-praziquantel tablet formulation, named TL-010 comprising (R)-praziquantel Form B with certain pharmaceutically acceptable excipients.

According to one embodiment, this new tablet formulation is capable of eliciting advantageous pharmacokinetic responses in human and animal subjects. In another embodiment, this new tablet formulation facilitates dissolution and improves flowability in the tablet manufacturing process.

Further, this tablet formulation includes 100-400 mg TL-010 as an active pharmaceutical ingredient.

One or more pharmaceutically acceptable excipients are added in various embodiments. For example, in one embodiment, by weight 2-10% of the tablet is disintegrant, 2-30% is binder, 2-40% is filler, and 0.3-3% is lubricant, 0.5-2% is surfactant and 0.1-0.8% is glidant. As described in the beginning of this Detailed Description, a multitude of substances may be suitably included as disintegrant, binder, filler, surfactant and stabilizer and lubricant. One example is to use sodium stearyl fumarate as lubricant, microcrystalline cellulose as filler, and CMC-Na as disintegrant. In a particular embodiment, the tablet formulation further includes povidone K30 as binder. By weight povidone K30 may constitute 1-4% of the tablet.

In embodiments, the relative percentage of formulation components (by weight) is as follows. Refer to following Table 2, as an active pharmaceutical ingredient (API), tablets of Group A and B contain (R)-praziquantel form B alone, while tablets of Group C to I are prepared by combined the (R)-praziquantel with one of other anthelmintic drug substances for human and animal use. According to a fixed ratio of the API mixture (TL-010:X), their relative percentage of the APIs and other ingredients (by weight) is as follows:

TABLE 2
The various tablet formulation compositions for TL-010 alone and its combinations (% w/w)

| | | Group | | | | | | | | |
|---|---|---|---|---|---|---|---|---|---|---|
| Ingredients | Function | A | B | C | D | E | F | G | H | I |
| Ratio of API combined (TL-010:X) | | 100 | 100 | 3:1 | 3:1 | 100:1 | 10:1 | 100:1 | 0.5:1 | 0.1:1 |
| TL-010 (R-praziquantel form B) | API | 50 | 70 | 50 | 50 | 50 | 50 | 50 | 50 | 50 |
| Microcrystalline cellulose PH-101 | Filler | 40 | 17.5 | 35.5 | 35.5 | 35.5 | 35.5 | 35.5 | 35.5 | 35.5 |
| Sodium Lauryl Sulfate | Surfacant | 1 | 1 | 1 | 1 | 1 | 1 | 1 | 1 | 1 |
| Povidone K30 | Binder | 2 | 2 | 2 | 2 | 2 | 2 | 2 | 2 | 2 |
| CMS-Na,Sodium Starch Glycolate (int) | Disintegrant | 3 | 3 | 3 | 3 | 3 | 3 | 3 | 3 | 3 |
| CMS-Na,Sodium Starch Glycolate (ext) | Disintegrant | 3 | 3 | 3 | 3 | 3 | 3 | 3 | 3 | 3 |
| Colloidal Silicon Dioxide | Glidant | 0.5 | 0.5 | 0.5 | 0.5 | 0.5 | 0.5 | 0.5 | 0.5 | 0.5 |
| Sodium Stearyl Fumarate | Lubricant | 0.5 | 3 | 5 | 5 | 5 | 5 | 5 | 5 | 5 |
| | Total | 100 | 100 | 100 | 100 | 100 | 100 | 100 | 100 | 100 |
| | Subjects | H, P | H, P | H, P | H, P | H | P | P | P | P |

Notes:
1. X denoted in the above groups: Groups A and B: (R)-praziquantel alone, Group C: Albendazole, Group D: tribendimidine, Group E: Ivermectin, Group F: Milbemycin Oxime, Group G: Moxidectin, Group H: Pyrantel pamoate and Group I: Fenbentel
2. Subject H: human, P: dog, cat, horse

The formulations of embodiments of the present invention can be prepared by a variety of techniques recognized in the art.

The manufacture of TL-010 tablets based on the tablet formulation of the various embodiments includes a series of steps. These steps are preparing TL-010 granulation, fluid bed drying, milling, lubrication blend, encapsulation, and bulk packaging.

The preparation of TL-010 granulation may be done in the following sequence. First, povidone K30 is mixed with water and dissolved using an overhead mixer. Second, TL-010 is milled with microcrystalline cellulose PH-101, Sodium lauryl sulfate and CMS-Na to break up any lumps. Third, the milled TL-010, microcrystalline cellulose PH-101, Sodium lauryl sulfate and CMS-Na are added into a high shear granulator and mixed. Fourth, the povidone and water solution are added to the blend. Fifth, the TL-010 granulation is blended for an additional period after the povidone and water solution have been completely added.

The fluid bed drying process may be performed on a fluid bed dryer with an inlet temperature of no more than 45° C. The milling process may be performed using a suitable miller such as Quadro Comil® or Airjet mill. The lubrication blend process may be conducted with the addition of an appropriate amount of CMS-Na (ext), colloidal silicon dioxide and sodium stearyl fumarate. The TL-010 granulation may be further blended at this point. For tablet, compress the final blend to 15.0 mm*6.0 mm tablets. Target tablet weight is 300 mg; and target hardness is 100N. The tablets may then be coated utilizing a film coating pan. The dose of 200-300 mg is yielded in an embodiment. To conclude the tablet manufacturing process, finished tablets may be packaged in secured, 45 ml HDPE bottle and stored at controlled room temperature.

Those skilled in drug research and drug making will appreciate that certain of the aforementioned steps may be modified or omitted, and additional steps may be included, without materially altering the outcome of the manufacturing.

According to some embodiments, the tablet formulation has good physical and chemical stability, thereby providing a shelf life of from 12 months to 36 months.

An exemplary composition and storage stability of the TL-010 formulation-containing tablets were prepared and examined, as listed in the Example 3 table 8 and Example 4. A representative batch of the TL-010/excipient formulation was prepared using routine wet formulation methods.

Capsule Formulation of (R)-Praziquantel

It is generally known that capsule formulations tend to facilitate the inclusion of high percentage of API with no or less pharmaceutical excipients. Capsules may allow for inclusion of a larger amount of binders, instead of fillers as used more in tablets. Where high percentage of API is desired and specific excipients are not known to be essential, capsule formulations are often adopted.

For the capsule formulation, the capsule shell may be made of hard gelatin in one embodiment. The shell may be clear or opaque, white or with color in various embodiments. The capsule is size 3 or 4 in an embodiment to yield a desired neat TL-010 active pharmaceutical ingredient (API) of 150 mg. Other sizes may be adopted in alternative embodiments. For example, to further improve the flowability of powder, the TL-010 granulation with excipients such as Sodium lauryl sulfate (SLS), is encapsulated using a suitable encapsulator into two-piece, size 1, gelatin capsule to yield a desired TL-010 dose of 150-300 mg and size 2 capsule to the 150 mg API with excipient.

Further, (R)-praziquantel, Form B, can be formulated for oral administration in capsules comprising 200 mg of (R)-praziquantel Form B. For these experiments, (R)-praziquantel, Form B with a D₉₀ particle size of between about 5 to 150 microns may be used. All inactive 200 mg ingredients may be GRAS and USP/NF excipients. The Strength manufacturing process may include wet granulation using a high shear mixer/granulator and filling into hard gelatin capsules.

Semi-Solid Oral Formulation for Veterinary Animals

The present disclosure also provides for a soft oral homogeneous anthelmintic veterinary formulation, for the treating, controlling and preventing of endo- and ectoparasite infections in warm blooded animals, in particular in companion animals such as cat and dog. This semi-solid oral composition or pasta formulation comprising consisting essentially of (R)-praziquantel alone or combined with at least one benzimidazoles (albendazole) or tribendimidine or a macrolide anthelmintic compound (milbemycin oxime or moxidectin), a solvent, which dissolves both the (R)-praziquantel and the benzimidazoles or tribendimidine, or milbemycin oxime or moxidectin and at least one filler, at least one surfactant, at least one flavoring, at least one thickener, at least one binder, at least one lubricant, at least one humectants, at least one preservatives agent, at least an opacifier and at least one antimicrobial agent.

In some embodiments, table 3 lists various compositions of soft oral anthelmintic veterinary formulations for pet animal (dog and cat). The compositions of an oral formulation contain the API (R)-praziquantel Form B and a benzimidazole (albendazole) or tribendimidine or macrollide compound or milbemycin oxime can be used for a warm blood animal such as a dog or a cat.

TABLE 3

| | Compositions | Amount (% w/w) |
|---|---|---|
| API | (R)-praziquantel Form B | 1-6% |
| | plus Albendazole | 1-6% |
| | or plus Tribendimidine | 1-6% |
| | or plus Milbemycin Oxime | 1-2% |
| | or plus Moxidectin | 0.1%(w/v) |
| Filler | Corn Starch | 1-40% |
| | Soy Protein Fines | 1-40% |
| thickeners | Hydroxypropyl cellulose | 1-30% |
| | microcrystalline cellulose (Avicel) | |
| | Corn starch | |
| | Xanthan gum | |
| | polyvinylpyrrolidone | |
| Flavoring | artificial beef (chicken, fish) flavor | 10-20% |
| Binder | Polyvinylpyrrolidone K30 | 2-8% |
| | Cross - linked polyvinylpyrrolidone | |
| | PEG 4000 | |
| Colorants | Titanium dioxide | 2% or none |
| Humectant | glycerol (water free) | 1-25% |
| | animal fats or fish oil | |
| Preservative | Benzyl alcohol | 1% |
| | Sodium benzoate | 0.40% |
| Antioxidant | butylated hydroxy anisole | 0.01-2% |
| | butylated hydroxytoluene | 0.15-5% |
| cyclodextrin | β-cyclodextrin | 0.5-40% |
| | hydroxethyl-3-cyclodextrin | |
| Buffer | 0.01M HCL | Add up to 100% |
| | | (pH 4-6) |
| viscosity | hydrogenated castor oil | 4% |
| modifier (oil | corn oil | |
| based only) | olive oil | |

In some other embodiments, the inventive soft oral formulation or pasta may be prepared, for example, by a process which comprises: dissolving the at least two different anthelmintic agents, e.g., (R)-praziquantel and macrolide compounds or benzimidazoles or tribendimidine, and thickening agent or agents into the solvent and forming a thickened solution; adding other non-active components such as artificial beef flavoring, zinc oxide, polyvinylpyrrolidone K30 and at least one compound selected from the group consisting of surfactants, preservatives or antioxidants and mixing well until a soft homogeneous pasta formulation is formed, wherein the method of manufacture does not include the addition of water, as shown in table 4.

TABLE 4

| Function | Compositions | Amount (% w/w) |
|---|---|---|
| API | R-praziquantel form B | 1.5 |
| | plus Albendazole | 25 |
| | or plus Tribendimidine | 25 |
| | or plus Milbemycin Oxime | 0.25 |
| | or pluse Moxidectin | 0.15 |
| Surfactant | sodium lauryl sulfate | 3 |
| Flavoring | artificial beef (chicken, fish) flavor | 25 |
| Thickener | corn starch | 20 |
| Binder | polyvinylpyrrolidone K30 | 6 |
| Solvent | propylene glycol | 6 |
| Lubricant | hydrogenated vegetable oils | 6 |
| Preservative | potassium Sorbate | 0.5 |
| Antimicrobial | benzyl alcohol | 4 |
| agent | | |
| Antioxidant | ascorbic acid | 1 |
| Opacifier | Zinc oxide | 1 |
| Filler | microcrystalline cellulose(Avicel) | Add up to 100% |

Another soft oral anthelmintic composition of embodiments of the present invention, in addition, is an aqueous based paste and may contain further veterinary acceptable inert ingredients, for example, solubilizers, colorants, humectants, buffers as shown in Example 5, table 11. Said veterinary acceptable ingredients are known to those skilled in the art of a veterinary formulation technology.

The water contents of the paste formulations of embodiments of the present invention are, for example 30% by weight, 20% by weight and in particular 10% by weight, in each case based on the weight of the entire formulation.

The solvents provided for in the inventive soft oral formulation are those polar solvent that will dissolve both the first anthelmintic agent (R)-praziquantel and at least one benzimidazoles (albendazole) or tribendimidine or one of macrolide compounds (milbemycin oxime or moxidectin). These solvents include, for example, propylene glycol, polyethylene glycol and glycerol.

The humectant may comprise more than one oil including, but not limited to, fat or fats, both natural and synthetic. A source for typical animal fats or oils are fish oil, chicken fat, tallow, choice white grease, prime steam lard and mixtures thereof. Humectants may typically present in the compositions at a concentration of about 1% to about 25% (w/w).

The thickeners contemplated by embodiments of the invention are well known to a practitioner of this art. Compounds which function as thickeners include, for example, celluloses, starches, natural gums, monothioglycerol, Synthetic polymers, such as polymers and copolymers of polyvinylpyrrolidone or (meth)acrylates, etc. Especially desired thickeners are microcrystalline cellulose (Avicel), hydroxypropyl cellulose, Xanthan gum and hydroxyethyl starch. Thickeners may be present in amounts of from about 1% to about 30%.

Opacifiers may be added to absorb and/or reflect certain light and/or energy of certain wavelengths and may thus enhance the stability of the formulations. Opacifiers include, for example, Zinc oxide or titanium dioxide and may be present in amounts from about 0.5 to 2.5%. Titanium dioxide is especially desired. These compounds are well known to practitioners of this art.

The resulting soft paste is then filled into the toothpaste tube-like, squeezable soft tube using a filling machine with different dosage units ranged from about 0.5 to 10 (g/mL) for different size animals. This cap-sealed toothpaste-like tube is made of plastic material with scored for volume measurement and one time disposable, with a total volume size from 0.5 mL to 10 mL. The soft tube offers the advantage e.g., of exact dosing and storage stability. Filling the exact dose of paste needed into a toothpaste tube like container and applying the paste directly into the mouth, assures that the animals are getting the exact dose. A prerequisite for the use of the soft oral paste formulation of embodiments of the present invention in medicinal paste tube especially for home-use is their excellent stability and shelf-life.

The soft oral compositions or pasta formulation of embodiments of the present invention are applied to warm blooded animals by means of a small and squeezable travel size toothpaste tube. Warm-blooded animals include, for example, all ruminants, equines, canines and felines. Especially desired are dogs, cats, cattle sheep, pigs, horses and the like. The amount of each of anthelmintic compounds is well known to a practitioner of this art. For pet animals such as dogs and cats, desired amounts of (R)-praziquantel include, for example, from about a 0.3 mg/kg to about 20 mg/kg of animal body weight, with a range of about 1 mg/kg to about 3 mg/kg or 1.5 mg/kg of body weight being especially. A most especially desired amount is about 3 mg/kg of animal body weight. Desired ranges for the anthelmintic macrolide compounds such as moxidectin include, for example about 0.1 to about 0.6 mg/kg, with about 0.3 to about 0.5 mg/kg being especially desired. Desired ranges for the anthelmintic benzimidazole compounds such as albendazole include 1 to 60 mg/kg of animal body weight. Desired ranges for the anthelmintic tribendimidine compounds include 1 to 60 mg/kg of animal body weight. A most especially desired amount for albendazole or tribendimidine is about 50 mg/kg of animal body weight. Desired ranges for the anthelmintic milbemycin oxime compounds include 0.1 to 40 mg/kg of animal body weight. A most especially desired amount is about 0.5 mg/kg of animal body weight. In one embodiment, dogs received the 2 grams soft paste compositions containing (R)-praziquantel form B and moxidectin or milbemycin oxime described in Tables 3, 4 and 11 to provide doses of 3 milligrams/kg (R)-praziquantel and 0.3 milligrams/kg moxidectin or 0.5 milligrams/kg milbemycin oxime per body weight of animal.

Embodiments of the anthelmintic semi-solid oral formulations according to embodiments of the present invention are listed in Tables 3, 4 and 11, respectively, for oil and aqueous based compositions of the paste formulations comprising (R)-praziquantel form B and other combined veterinary medicines as well as the inert ingredients.

In one embodiment, the oral veterinary composition is a paste composition. In another embodiment, the oral veterinary composition is a tablet or chew or granular dosage forms, as shown in Tables 2, made using a forming machine. The soft oral compositions or pasta formulations of embodiments of the present invention are suitable for controlling pathogenic endoparasites in warm-blooded animals such as, companion animals, in particular dogs or especially cats; they have a favorable safety profile to warm-blooded species, in particular, removal of (S)-praziquantel, a 50% of component responsible for a bitter taste and bad smell and potential toxic component in racemic praziquantel used in the commercial combination veterinary medicine. They are effective against all or individual development stages of the pests. Pathogenic endoparasites include cestodes, trematodes, nematodes and acanthocephala.

Liquid Compositions and Solid Formulations Containing (R)-Praziquantel Form B for Aquaculture and Fish

Fish, most economically important food fisheries in the world, like other vertebrates, suffer production issues of health, welfare, and feed efficiency when infected with helminth parasites. Both larval and adult forms of cestodes and trematodes parasitize fish. In production aquaculture, it becomes necessary to treat fish to prevent economic losses. In other cases, fish may contain larval cestodes or trematodes that are infectious for humans, thereby requiring treatment. In addition, aquarium or ornamental fish may require treatment.

A parasitically effective amount of the (R)-praziquantel Form B may be present in an acceptable formulation to fish.

Generally, the (R)-praziquantel Form B formulations for use in eliminating fish ecto-parasite such as monogeneans by bath treatment in the form of liquid compositions, in pure form, and/or as a solid active substance (for example, in a specific particle size (R)-praziquantel Form B between 0.1-150 microns, more between 0.5-10 microns). For example, by placing the fish into a “medicinal bath” and keeping them there for a period of time (minutes to several days, preferable between 1 hour to 48 hours, more preferable between 5 to 24 hours), for example, when being transferred from one net pen or breeding basin to another, for example, the net cages, entire ponds, aquaria, tanks or basins in which the fish are kept. The drug substance may be prepared or formulated in any suitable form, such as powder, granulate, solution, emulsion, micro/nano emulsion, emulsifiable concentrate, suspension, nanosuspension, or suspension concentrate, tablet or the simply pure (R)-praziquantel Form B itself. For example, the formulation may be suitable for dispersing the (R)-praziquantel Form B in water containing the fish to a final concentration of 1-50 mg/L for up to 96 hours, preferable at 10 mg/L between 1 hour to 48 hours, more preferable at 5-25 mg/L between 6 hours to 24 hours.

For endo-parasites in fish, oral administration with formulations comprising (R)-praziquantel Form B is a desired way. It may take the either of following forms, for example, 1) feed additives: pellet, powders, premixes, granulates, solutions, emulsions, micro/nano emulsions, emulsifiable concentrates, suspensions, nanosuspensions or suspension concentrates are mixed homogeneously as feed additives with the feed; or 2) pill: powders, premixes, granulates, solutions, emulsions, micro/nano emulsions, emulsifiable concentrates, suspensions, nanosuspensions or suspension concentrates are administered in the form of pills, the outer coat of which can consist e.g. of fish feed compositions which cover the active substance completely.

In an embodiment, the (R)-praziquantel Form B formulation may comprise at least one of the adjuvants which are conventionally used in formulation technology, such as solvents, excipients, solubilizers, etc. The formulations may be prepared in a manner known per se, typically by mixing, granulating and/or compacting the (R)-praziquantel Form B with solid or liquid carriers, where appropriate with the addition of further adjuvants, such as emulsifiable or dispersing agents, stabilizers, colorants, antioxidants and/or preservatives.

In an embodiment, medicated fish feed may be used in the form of fish feed granules or pellets which have a coating comprising the (R)-praziquantel. In an embodiment, the fish food includes the (R)-praziquantel and at least one of corn starch, pregelatinized corn starch, protein, nutrients, vegetable oil and/or fish oil. Or alternatively, commercially available fish pellets or granules may be coated with a pre-mix containing the (R)-praziquantel and one or more suitable excipients such as a starch, fumed silica (Aerosil®), microcrystalline cellulose, lactose or the like.

The (R)-praziquantel Form B may be in the form of medicated fish feed or given by intubation via the esophagus. For example, the medicated fish feed may be in the form of fish feed granules or pellets which have a coating comprising (R)-praziquantel Form B. To coat the pellets, 25 g of gelatin (Davis Gelatine, New Zealand) was dissolved in 625 ml of hot tap water (50° C.) on a magnetic heater/stirrer. Once dissolved, this gelatin solution was poured into a rotating cement mixer containing 25 kg of pellets and the necessary quantity of (R)-praziquantel. Mixing continued for a further 5 min to ensure a homogeneous coverage of gelatin and (R)-praziquantel. Pellets were then removed from the mixer, spread in a thin layer and placed in a cool room at 8° C. to allow the gelatin to set. Once set, pellets were returned to feed bags until use.

The vehicle for intubation was prepared by heating 60 g of food starch (Solamyl) in 1 L H₂O to 100° C. and after cooling down, the vehicle was mixed with (R)-praziquantel Form B at 5 mg/kg, 50 mg/kg and 200 mg/kg body weight. The dose regiment is between 5 to 200 mg/kg body weight, single or daily doses for 3 up to 20 days, preferable at 200 mg/kg body weight single dose, more preferable between 50 to 100 mg/kg body weight single daily dose for 3 to 5 consecutive days.

The (R)-praziquantel Form B may be formulated in combination with other active agents that is capable of treating parasitic infections in fish.

Representative preparation methods, formulation compositions, physicochemical Characterization and stability of API and drug product, and treatment results in accordance with embodiments of the invention are presented in the following examples.

### EXAMPLES

### Example 1. Manufacture of (R)-Praziquantel Form B

A scale-up procedure was developed, and process parameters were identified to exclusively produce anhydrous Form B of (R)-2-(cyclohexanecarbonyl)-2,3,6,7-tetrahydro-1H-pyrazino[2,1-a] isoquinolin-4(11bH)-one.

The solution containing about 1.5 kg (R)-praziquantel in ethyl acetate was concentrated to lower volume (around 5 v/w) in a 50 liters jacketed reactor under vacuum (≤−0.08 MPa), maintaining the jacket temperature between 25° C. to 45° C. The resulting mixture was swapped with isopropanol twice (4.6 Kg/each) to ˜3 v/w. While the batch temperature was increased to 40° C.˜50° C., n-heptane (21 Kg) was added to the batch. The obtained mixture was agitated for about 1 hour. The batch temperature was cooled to 0˜5° C. over about 3 hours, and the mixture was agitated between 0˜5° C. for about another 2 hours. The mixture was filtered by a filter, and the cake was washed with n-heptane (5.1 Kg). The isolated wet (R)-praziquantel was dried under vacuum (≤−0.08 MPa) oven at 35±5° C. for at least 10 hours.

The white solid was sampled for XRPD, KF and HPLC. HPLC purity and weight assay testing. Weight: 1.2 Kg, Assay: 100%, HPLC Purity: 99.9% (214 nm), Chiral Purity: 100%.

Recrystallization with isopropanol/n-Heptane and drying under vacuum at 35±5° C. for at least 10 hours of drying are critical step for an efficient removal of unbound water in (R)-2-(cyclohexanecarbonyl)-2,3,6,7-tetrahydro-1H-pyrazino[2,1-a] isoquinolin-4(11bH)-one Form B, which ensure high purity of the active pharmaceutical ingredient meet the industrial GMP criteria.

The XRPD spectrum of (R)-praziquantel Form B is displayed in FIG. 1 with XRPD data listed in Table 5. DSC/TGA spectrum is displayed in FIG. 2.

TABLE 5

| Index | Angle | d Value | | Rel. Intensity |
|---|---|---|---|---|
| 1 | 7.217° | 12.23855 | Å | 8.00% |
| 2 | 8.619° | 10.25079 | Å | 100.00% |
| 3 | 13.069° | 6.76896 | Å | 3.70% |
| 4 | 13.754° | 6.43332 | Å | 3.90% |
| 5 | 14.369° | 6.15921 | Å | 5.40% |
| 6 | 15.328° | 5.77580 | Å | 18.80% |
| 7 | 15.981° | 5.54139 | Å | 11.00% |
| 8 | 16.900° | 5.24193 | Å | 4.60% |
| 9 | 17.856° | 4.96336 | Å | 32.30% |
| 10 | 18.208° | 4.86845 | Å | 9.50% |
| 11 | 19.262° | 4.60423 | Å | 13.00% |
| 12 | 20.062° | 4.42246 | Å | 10.50% |
| 13 | 20.914° | 4.24412 | Å | 4.80% |
| 14 | 22.274° | 3.98805 | Å | 16.60% |
| 15 | 23.347° | 3.80707 | Å | 1.40% |
| 16 | 24.080° | 3.69277 | Å | 9.90% |
| 17 | 24.783° | 3.58965 | Å | 8.90% |
| 18 | 25.785° | 3.45241 | Å | 4.40% |
| 19 | 27.794° | 3.20722 | Å | 7.30% |
| 20 | 31.210° | 2.86357 | Å | 2.40% |
| 21 | 34.530° | 2.59541 | Å | 2.10% |

### Example 2. Physicochemical Characterization and Stability of Anhydrous Form B of (R)-Praziquantel

Form B is reliably manufactured by the industrial crystallization process with more than 99.0% purity and with impurities not more than 0.15% and water content under ˜0.5% or less, the detailed test results were listed in the Table 6.

TABLE 6

| Test Items | Acceptance Criteria | Initial (0 M) | 18 M |
|---|---|---|---|
| Appearance | White to off-white solid | White solid | White solid |
| XRPD Characterization | Conform to Form B | Conforms | Conforms |
| Purity | ≥98.0% | 99.90% | 99.90% |
| Assay(Anhydrous basis) | 97.0%-103.0% | 99.80% | 101.00% |
| Related Substances | Largest single | RRT = 0.60: 0.04% | RRT = 0.60: 0.03% |
| | impurity ≤0.10% | | |
| | Total impurities ≤2.0% | 0.10% | 0.11% |
| Chiral Purity | ≥99.0% | 100.00% | 100.00% |
| Water Content | Report the result | 0.50% | 0.27% |

Water Content by Karl Fischer Method

Accurately weigh about 0.1-0.5 g of sample of Form B and transfer it to KF titrator container (aldehyde ketone Volumetric method), measure the samples according to USP<921>.

Differential Scanning Calorimetry (DSC)

Weigh approximately 2-3 mg of the compound into a non-hermetically sealed aluminum pan and crimp the lid. Heat the sample from 25° C. to 300° C. at the rate of 10° C./min. Integrate all endothermic and exothermic events and report onset, peak, and heat of fusion/crystallization.

Thermo-Gravimetric Analysis (TGA)

Weigh approximately 10 mg of the compound into a platinum pan. Heat the sample from ambient to 300° C. at the rate of 10° C./min with sample purge flow at 60 mL/min and balance purge flow at 40 mL/min.

Present the data as a plot of weight % change versus temperature. Determine the weight loss at temperatures where endothermic and exothermic events were found in DSC or as a default loss of weight between ambient and 120° C.

For (R)-praziquantel Form B, a melting point was observed at 112.5° C. DSC data indicated a strong and sharp endotherm at 109° C. (onset) corresponding to weight loss in the TGA, followed by a strong endotherm, presumably the melt of the Form B, at 112° C. The DSC thermogram for the anhydrous crystalline PZQ (FIG. 2) shows no phase transitions until melting at 109° C. to 112° C. Desolvation and dehydration were complete on the TGA within 15 minutes at 100° C. and within 40 minutes at 60° C. About 1.5% weight loss was observed when heated from room temperature up to 90° C. on the TGA thermogram, which is attributed to decomposition of the volatiles.

In sum, Form B of example 1 is an anhydrous crystalline solid with about 0.1 moles of water (<0.5% w/v), which had a DSC thermogram exhibiting an endotherm between about 105° and 115° C. and an endotherm at about 112° c. It is also a slightly hygroscopic.

The active pharmaceutical ingredient (API) of Form B was stored for 18 months under different storage conditions: 25° C. and 60% relative humidity, 30° C. and 65% relative humidity, and 40° C. and 75% relative humidity, in a sealed and open condition. The results show that good stability under various storage conditions over time. In some embodiments, under various storage conditions the anhydrous API provided herein can be stable and water content controlled under 0.5% and monitored by KF for at least, or at least about, 3 months, 6 months, 9 months, 12 months, 18 months, 24 months and 36 months. For example, under storage conditions of 25° C. and 60% relative humidity, the tablets and API provided herein can be stable for at least, or at least about, 3 months, 6 months, 9 months, 12 months and 18 months.

The long-term stability and water content of (R)-praziquantel form B API provided herein at 25° C. and under 60% relative humidity demonstrated in the results presented in above Table 6.

The Accelerated Solid State Stability of the Anhydrate (Form B) and Hemihydrate (R)-Praziquantel in an Open and Closed Condition

At 45° C. and 75% relative humidity under open condition, accelerated stability was conducted for TL-010 anhydrate in comparison to the (R)-praziquantel hemihydrate, the result showed an impurity occurred in the hemihydrate at the end of 3 months storage period. The results presented in below Table 7.

The hemihydrate (R)-praziquantel was prepared according to the reported method. (refer to Myer, et al).

The accelerated solid-state stability of the anhydrate and hemihydrate (R)-praziquantel was determined by storing approximately 1.0 g of the material in a glass dish at a) the anhydrate at 45° C./75% (RH), closed, for 3 months. and b) 45° C./75% Relative Humidity (RH), open exposure condition, for 3 months. The material was assayed by KF, TGA and HPLC for final content and degradation products.

Results: a) Closed condition: no significant degradation observed (HPLC assay 99.9% initial). The KF test of the anhydrate form shows that the water uptake does not exceed 0.3%; b) Opened condition: no significant degradation observed (HPLC assay 99.9% initial) for the anhydrate. However, there are two impurity peaks found for the hemihydrate, with the percent total impurities being over 0.3%.

Meanwhile, the KF test of the anhydrate form shows that the water uptake is increased about 2.5% from initial 1.3% and none of impurity peak found when left open for 3 months at 75% RH, temperature 45° C. (Table 7). This indicates that the anhydrate form is stable even at the accelerated conditions and does not degrade significantly.

TABLE 7

| | | | Storage period (month) | |
|---|---|---|---|---|
| Crystal form | Storage Condition | Test method | T0 | T3 |
| Anhydrate | Sealed, 45° C., RH75% | Water content (KF) | 0.24% | 0.32% |
| | | Impurities by HPLC | ND* | RRT 0.6: 0.04% |
| | Open, 45° C., RH75% | Water content (KF) | 1.30% | 2.50% |
| | | Impurities by HPLC | ND | RRT 0.6: 0.04% |
| Hemihydrate | Open, 45° C., RH75% | Water content (KF) | 2.80% | 2.50% |
| | | Impurities by HPLC | ND | RRT0.6: 0.17%, |
| | | | | RRT0.9: 0.13% |

*Not detected

Overall, there has been discovered surprisingly an extremely stable anhydrous crystalline (R)-praziquantel Form B, which retains a desired physical appearance and analytical chemical properties over a longer period of time. Because of the unusually high purity and extreme stability of anhydrous crystalline (R)-praziquantel it is favorably suited to be used in formulation development as a final drug product on an industrial scale.

### Example 3. Composition, Stability and Dissolution Profiles of Tablet Containing Form B

The following formula (seen Table 8) was used to prepare the finished dosage form, i.e., a tablet providing 150 mg of TL-010.

TABLE 8

| Function | Ingredient | % w/w | mg |
|---|---|---|---|
| | API | 50 | 150 |
| Filler | Microcrystalline cellulose PH-101 | 40 | 120 |
| Surfacant | Sodium Lauryl Sulfate | 1 | 3 |
| Binder | Povidone K30 | 2 | 6 |
| Disintegrant | CMS-Na, Sodium Starch Glycolate (int) | 3 | 9 |
| Disintegrant | CMS-Na, Sodium Starch Glycolate (ext) | 3 | 9 |
| Glidant | Colloidal Silicon Dioxide(Aerosil@200 | 0.5 | 1.5 |
| | Pharma) | | |
| Lubricant | Sodium Stearyl Fumarate | 0.5 | 1.5 |
| Total | | 100 | 300 |

In addition to the therapeutic advantages of the TL-010 formulations provided herein, these tablets formulations also show good stability under various storage conditions over time. In some embodiments, under various storage conditions the TL-010 formulations provided herein can be stable for at least, or at least about, 3 months, 6 months, 9 months, 12 months, 18 months, 24 months, 36 months, or 48 months. For example, under storage conditions of 25° C. and 60% relative humidity, the tablets and TL-010 formulations provided herein can be stable for at least, or at least about, 3 months, 6 months, 9 months, 12 months, and 18 months.

In another example, under storage conditions of 40° C. and 75% relative humidity, the tablets and TL-010 formulations provided herein can be stable for at least, or at least about, 3 months, 6 months.

In some embodiments, the stability of the tablets and TL-010 formulations provided herein is determined by measuring the dissolution rate of the stored tablet and/or TL-010 formulations. Any of a variety of dissolution methods provided herein or otherwise known in the art can be performed to evaluate the stability of TL-010 tablet formulations. Dissolution measurements are in vitro methods known in the art as a strong indicator of in vivo pharmacokinetic performance. Accordingly, the stability of the TL-010 tablet formulations as measured by dissolution methods will be indicative of the in vivo Tₘₐₓ and AUC values of a subject when the TL-010 formulations after storage, for example, under the above-exemplified conditions for the indicated amount of time. Typically, a dissolution level indicative of an acceptable level of stability is a dissolution of at least, or at least about 80%, 85%, 90%, 91%, 92%, 93%, 94%, 95%, 96%, 97%, 98%, 99%, or more, of the TL-010 in the tablets provided herein. Any of a variety of dissolution methods provided herein or otherwise known in the art can be performed to determine the stability of TL 010 formulations. For example, dissolution can be determined according to the pharmacopeial dissolution method specified in USPII.

The 300 mg TL-010 tablet formulation of Example 3 was stored for 24 months under two different storage conditions: 25° C. and 60% relative humidity, and 40° C. and 75% relative humidity. Results show that the tablet did not appreciably change over the duration of the 24-month period. The dissolution of the TL-010 formulations at 40° C. and 75% relative humidity did not appreciably change over the initial 6-month period. The dissolution analysis was performed according to the pharmacopeial dissolution method specified in USPII using Apparatus 2 (paddles) with water as a solvent and a specification of Q˜75% of label claim in 30 minutes. The level of total impurities in each formulation, as determined by HPLC, was less than 0.1% over the duration of the 24-month period. Finally, the percent of TL-010 in each sample, as determined by HPLC, showed no appreciable degradation over the 24-month period.

Table 9 shows the long-term stability and dissolution profiles of the TL-010 tablet formulation comprising (R)-praziquantel form B at 25° C. and under 60% relative humidity.

TABLE 9

| Test items | | Specification | 0 M | 18 M |
|---|---|---|---|---|
| Appearance | | White to off-white biconvex | White biconvex | White biconvex |
| | | capsule shape tablet | capsule shape tablet | capsule shape tablet |
| Identification | | Retention time of main peak in | Complies | NA |
| | | sample solution should be | | |
| | | consistent with that in working | | |
| | | standard solution | | |
| Assay | | Should be between 90.0% and | 97.70% | 101.60% |
| | | 110.0% of the labeled amount | | |
| | | of TL-010 | | |
| Related | Single | Not more than 0.1% | <0.05% | RRT 0.84: 0.06% |
| Substances | maximum | | | RRT 0.90: 0.06% |
| | unknown | | | |
| | impurity | | | |
| | Total | Not more than 1.0% | <0.05% | 0.12% |
| | Impurities | | | |

| | | | |
|---|---|---|---|
| Dissolution | Not less than 75% (Q) of the | 1)92.8% | 1)91.3% |
| | labeled amount of TL-010 is | 2)94.0% | 2)91.4% |
| | dissolved in 60 minutes. | 3)93.2% | 3)90.1% |
| | | 4)93.1% | 4)91.0% |
| | | 5)90.4% | 5)92.5% |
| | | 6)92.4% | 6)91.1% |
| | | Mean (n = 6): 93% | Mean (n = 6): 91% |

As can be seen from the tables 6 and 9, the unusual stability of the anhydrous crystalline (R)-praziquantel results in highly reproducible batches and better storage characteristics. During the storage period of the API and the tablet formulation, there is neither chemical degradation nor enantiomer conversion were determined by the standard analytical methods, which ensure the consistent results in solubility, dissolution and pharmacokinetic studies in human and animals, further providing solid evidence for the anhydrous form B of (R)-praziquantel as an excellent candidate for scale-up manufacture process and clinical development.

### Example 4. An Alternative Composition of Tablet Containing Form B

The following formula (seen Table 10) was used to prepare a tablet providing 150 mg of TL-010.

TABLE 10

| Ingredient | Function | Amount (mg) |
|---|---|---|
| R-praziquantel form B | API | 150 |
| Microcrystalline cellulose PH-101 | Filler | 112.5 |
| Sodium Lauryl Sulfate | Surfacant | 3 |
| Povidone K30 | Binder | 6 |
| CMS-Na, Sodium Starch Glycolate (int) | Disintegrant | 9 |
| CMS-Na, Sodium Starch Glycolate (ext) | Disintegrant | 9 |
| Colloidal Silicon Dioxide | Glidant | 1.5 |
| Sodium Stearyl Fumarate | Lubricant | 9 |
| | Total | 300 |

### Example 5. Soft Oral Compositions or Pasta Formulation Containing (R)-Praziquantel Form B

Table 11 lists compositions of a soft oral anthelmintic veterinary formulation for pet animal (dog and cat). The compositions of an oral formulation contain the API (R)-praziquantel Form B and a benzimidazoles (albendazole) or tribendimidine or macrolide compound or milbemycin oxime can be used for a warm blood animal such as a dog or a cat.

TABLE 11

| Function | Compositions | Amount (% w/w) |
|---|---|---|
| API | R-praziquantel form B | 1.5 |
| | plus Albendazole | 25 |
| | or plus Tribendimidine | 25 |
| | or plus Milbemycin Oxime | 0.25 |
| | or pluse Moxidectin | 0.15 |
| Filler | microcrystalline cellulose (Avicel) | 25 |
| Colorant | titanium dioxide | 2 |
| Solublizer | HPβCD | 3 |
| Flavoring | artificial beef (chicken, fish) flavor | 20 |
| Binder | polyvinylpyrrolidone K30 | 6 |
| Humectant | propylene glycol | 6 |
| Preservative | potassium Sorbate | 0.3 |
| Buffer | 0.01M Citret buffer | Add up to 100% |

As an example, the orally applied, homogeneous aqueous suspension paste, may be prepared by the following process:
1. Prepare for a solution in 0.01 M Citrate buffer and microcrystalline cellulose with propylene glycol or HBβCD.
2. Dissolve (R)-praziquantel form B and albendazole (or tribendimidine), and Milbemycin Oxime or at least one macrolide anthelmintic compound or compounds and the thickening and filler agents or agents into the solution and mix well at ambient temperature.
3. Add other non-active components such as artificial beef flavoring, titanium dioxide, polyvinylpyrrolidone K30 and least one compound selected from the group consisting of surfactants, preservatives or antioxidants and mix until homogeneously dispersed.
4. Finally add the remaining the buffer to the mixture in a blender and mix well to yield a soft homogeneous paste.
5. The resulting semi-solid formulation is then filled into a toothpaste tube-like, squeezable soft tube using a filling machine with different dosage units ranged from about 0.5 to 10 (g/mL) for different size animals.

### Example 6. A Pilot Clinical Pharmacokinetics and Pharmacodynamics Study of (R)-Praziquantel Form A

(reported in CN104327077A) in Treatment of Schistosomiasis Japonica One exploratory clinical study was conducted on a single dose of 30 mg/kg of (R)-praziquantel in 8 patients with chronic schistosomiasis japonica. These adult patients (4 males and 4 females) were selected by a stool miracidial hatching method from an endemic area and were admitted to a local hospital. During hospitalization, standard diet was provided, alcohol was prohibited, irrelevant medications were stopped to use. Three out of the eight patients were diagnosed with slight hepatomegaly and two with mild ascites. Most of clinical chemistry tests showed no abnormalities in hepatic and renal function. (R)-praziquantel in white crystalline powder was prepared in gelatin capsules (200 mg/capsule, Size 2 capsule) as neat API (Form A). On day 1, all patients were given orally with (R)-praziquantel at a 30 mg/kg single dose followed by about 200 mL drinking water.

Clinical observation: each patient was monitored closely after drug administration with a focus on the patients' complaints and signs. All patients well tolerated to the treatment with mild and transient side effects. ECG examinations before, 2-hour and 24-hour post dose showed no remarkable changes. Clinical chemistry tests of hepatic and renal function in blood and urine were also unchanged before and after the drug treatment.

Pharmacokinetics: Blood samples were collected via elbow vein at pre-dose and 1, 2, 4, 6-, 8-, 12- and 24-hours post dose, respectively. Blood samples were processed to obtain serum and stored in −40° C. freezer until analysis. Serum concentrations were measured using a reversed phase high performance liquid chromatography (HPLC) developed in house. At 60 minutes after oral ingestion of the capsules, the average serum concentration of (R)-praziquantel reached 0.13 μg/mL. At two hours post dose, the average serum concentration was measured to be 0.15 μg/mL, and at eight hours 0.09 μg/mL. The pharmacokinetic result of the capsule treatment with this study is listed in the table 12.

Therapeutic effect: Stool miracidial hatching tests for 3 successive times were conducted for each patient 6 months after the treatment. The 100% negative conversion rates of stool miracidial hatching tests confirmed that the 8 patients were all cured.

### Example 7. Reported Pharmacokinetic Studies on (R)-Praziquantel Oral Dispersible Tablet (ODT)

Two pharmacokinetic studies have been reported in healthy adult males (Bagchus et al, 2019 Relative Bioavailability of Orally Dispersible Tablet (ODT) Formulations of Levo- and Racemic Praziquantel: Two Phase I Studies Clin Transl Sci. 2019 January; 12(1):66-76. Merck KGaA, Darmstadt, Germany, hereafter “Bagchus 2019”).

In this study, levo-praziquantel (i.e. R-PZQ)) was formulated as an orally dispersible tablet (ODT) formulation and was administered orally to 17 healthy adult subjects (Group D) at 30 mg/kg. Plasma samples were collected predose and 0.5, 1, 1.5, 2, 2.5, 3, 3.5, 4, 4.5, 5, 5.5, 6, 8, 12, 16, and 24 hours postdose. R-PZQ concentrations in plasma were determined by using validated enantioselective LC/MS assays. The resulting values of pharmacokinetic parameters are listed in the Table 12. The ODT formulation included 150 mg of active ingredient R-PZQ and certain undisclosed pharmaceutically acceptable excipients as well as mannitol and sweeteners for taste masking. For a comparison at the same oral dose of 30 mg/kg level, the pharmacokinetic results among the studies with the ODT, the capsule in Example 6 and the TL-010 table in Example 8 below are summarized in the Table 12 below.

### Example 8. Pharmacokinetic Studies of the Current TL-010 Tablet of (R)-Praziquantel of Example 3 in Human

A first study depicted in FIG. 3 shows average changes in plasma concentrations over time in three groups of human healthy subjects to whom were administered a single dose of the TL-010 tablet formulation at different dose levels. The three plasma drug concentrations-time curves of this graph represent three different dose groups of subjects: A, 20 mg/kg; B, 30 mg/kg; C, 40 mg/kg. As shown in FIG. 3, a dose-proportional increase in (R)-praziquantel exposure (AUCs and Cₘₐₓ) was observed, suggesting a linear kinetics of TL-010 over the dose range of 20 to 40 mg/kg in the healthy subjects.

In another pharmacokinetic study, two groups of 23 human healthy subjects under fasting conditions were included under a crossover study design. One group (Group I) received TL-010 tablet at 20 mg/kg, while the other group (Group II) received the reference drug Biltricide® tablet at 40 mg/kg. Plasma concentrations of (R)-praziquantel were determined by a chiral LC/MS/MS method at pre-dose and 0.25, 0.5, 1, 1.5, 2, 3, 4, 6, 8, 12 and 24 hours post dose. FIG. 4 is a plot of plasma (R)-praziquantel levels over time summarizing this study. As shown in FIG. 4, the maximum plasma (R)-praziquantel level was reached at 1.5 hr postdosing for both groups. However, surprisingly as shown in the plot, averaged Cₘₐₓ is significantly higher, an approximately 75% increase in TL-010 tablet Groups than what was found in the same subjects dosed with the reference compound Biltricide®.

To compare with the previous results from Example 7, the report by Bagchus et al 2019 where an oral dispersible tablet (ODT) formulation was used and from Example 6 in which a neat (R)-praziquantel powder in capsule without excipients was used, a pharmacokinetic comparison was summarized in the table 12 for three (R)-praziquantel oral formulations (the current TL-010 tablet, ODT and the capsule) at the same single oral dose of 30 mg/kg level under fed condition. AUC of TL-010 tablet is significantly higher (approximately 125% and 19% increase) than those of the capsule and the ODT. AUC values are computed over a time period of zero to infinity. The value of Tₘₐₓ is shorter with the TL-010 tablet than those with the ODT and the capsule formulations. Overall, a faster oral absorption and a greater plasma exposure of (R)-praziquantel are seen with the TL-010 tablet when compared to the ODT and the capsule formulations.

TABLE 12

| PK Parameters | Tablet | ODT | Capsule |
|---|---|---|---|
| Example No. | 8 | 7 | 6 |
| Subject | health | health | patients |
| Oral dosage(mg/kg) | 30 | 30 | 30 |
| Subjects (n) | 24 | 17 | 8 |
| AUC (ng/mL · hr) | 2760 | 2324 | 1229 |
| Tmax (hr) | 1.5 | 3 | 2.8 |
| T½ (hr) | 4 | 3 | 5 |

The same trend was observed when plasma exposure (AUC) of the TL-010 tablet at 40 mg/kg group was compared to results of the (R)-praziquantel ODT at the same dose group under fast condition reported by Bagchus 2019, as shown in the Table 13. Unexpectedly, the mean AUC value of the TL-010 tablet is about 3.68-fold higher than that with the ODT in Group A.

TABLE 13

| | | | | | | | | Geometric mean | | |
|---|---|---|---|---|---|---|---|---|---|---|
| | | | | | | | Case | AUC | Tmax | T½ |
| Group | Reference | Study No. | Study design | Oral dosage | | Subjects | number | ng · hr/mL | hr | hr |
| A | Orally | RMR | Open, random, | 150 | mg | Healthy adult | 35 | 506 | 2 | 3 |
| | Dispersible | 200661-001 | crossover, single | 40 | mg/kg | 18-55 years | | | | |
| | Tablet | | oral dose, | | | old | | | | |
| | | | fast condition | | | | | | | |
| B | TL-010 | TL-010- | Open, random, | 150 | mg | Healthy adults | 22 | 2370 | 1.5 | 5 |
| | tablet | 2018TL003 | crossover, single | 40 | mg/kg | 19-48 years | | | | |
| | | | oral dose, | | | old | | | | |
| | | | fast condition | | | | | | | |

### Example 9. Pharmacokinetic Studies of the Current (R)-Praziquantel Tablets of Example 3 in Dogs

A pilot cross-over pharmacokinetic study was performed in dogs dosed orally with TL-010 tablets of the present disclosure. Pharmacokinetic and relative bioavailability study of (R)-praziquantel (R-PZQ) following oral administration of TL-010 (150 mg/tablet) versus half of commercial PZQ tablet (Biltricide®, 600 mg/tablet containing the same amount of (R)-praziquantel (150 mg/tablet) and S-isomer) in two male beagle dogs. Table 14 summarizes the resulting PK values for these groups. The values of Tₘₐₓ are shorter, Cₘₐₓ and AUC are also higher for plasma R-PZQ in the dogs dosed with the TL-010 tablet than those with the racemic PZQ tablet. The results show that racemic PZQ have much higher blood levels for S-PZQ and its metabolite S-hydroxyl isoquolonine, the potential components may elicit isomer-drug interaction and raise safety concerns when dosed with other drugs in combination therapy. While dosing the dogs with R-PZQ alone, there are neither S-isomer and its metabolite, nor conversion from the S-isomer or its metabolite to R-PZQ detected, indicating a relatively safer profile to the animals.

TABLE 14

| PK parameters | TL-010 tablet | Biltricide ® |
|---|---|---|
| Tmax (hr) | 0.4 | 2 |
| Cmax (ng/mL) | 66 | 23 |
| AUC (hr*ng/mL) | 39 | 31 |

These resulting PK values shown in examples 6-9, especially the increased Cₘₐₓ and AUC, indicate a relatively greater bioavailability for the R-PZQ tablet formulation according to the present disclosure. Consequently, these tablets are capable of sustaining better therapeutic actions in a subject (a patient or an animal). Note that the drug formulations in these previously reported studies were different. Bagchus 2019 in Example 7 used an oral dispersible tablet (ODT) formulation that included 150 mg of active ingredient (R-PZQ) and certain undisclosed pharmaceutically excipients as well as mannitol and sweeteners for taste masking, while Example 6 used the 200 mg of the neat (R)-praziquantel Form A in a capsule without any excipients. Therefore, compared to the 100% Form A in capsule used in Example 6 and the ODT tablets in example 7, the current tablet formulation comprising the anhydrous Form B with the excipients may be administered to a subject in need, thereby eliciting desirable pharmacokinetic responses in the subject. Whilst such desirable PK responses are unexpectedly and surprising results, it is conceivable that certain compositions and amount of the excipients such as disintegrant CMS-Na (Sodium Starch Glycolate) and lubricant and sodium stearyl fumarate, binders such as microcrystalline cellulose, povidone K30 may play some roles to favorably interact with the (R)-praziquantel molecule.

### Example 10. Therapeutic Indications

One embodiment of this disclosure provides methods for treating diseases such as schistosomiasis japonica, schistosomiasis haematobium, schistosomiasis mansoni, clonorchiasis, opisthorchiasis, paragonimiasis, sparganosis mansoni, fasciolopsis, echinococcosis, taeniasis, cysticercosis including neurocysticercosis, etc.

These methods comprise administering the TL-010 tablets of this disclosure to a patient suffering from a parasitic infection condition or a cytokine-mediated disorder. The dosing may be once, twice or three times daily, with one or more tablets per intake. According to a particular embodiment, the total daily intake is at least 1500 mg to a human subject or at least 1 mg/kg to a warm blood animal like dog or cat.

The total daily intake amount may vary, depending on the subject profile, including among other things the patient's demographic characteristics, physiological and genetic conditions, and disease prognosis. For example, a child or a senior human being may be given a lower amount daily than that given to an ordinary adult.

The anti-parasitic activity of (R)-praziquantel is demonstrated in various in vivo animal models, as well as in vitro culture studies with various adult parasitic worms, including schistosomes, liver fluke, tape worm, and others. Those data indicate that (R)-praziquantel may be an effective agent for treating parasitic infections caused by diseases such as schistosomiasis japonica, schistosomiasis haematobium, schistosomiasis mansoni, clonorchiasis, opisthorchiasis, paragonimiasis, sparganosis mansoni, fasciolopsis, echinococcosis, taeniasis, cysticercosis, etc.

The human clinical findings after treatment with (R)-praziquantel have been consistent with the anti-schistosome effects observed in the laboratory animals. Pilot open clinical trials with oral (R)-praziquantel have been undertaken with patients afflicted with schistosomiasis japonica, schistosomiasis mansoni, schistosomiasis hematobium, choloanchiasis sinica and opisthorchiasis.

One of the clinical beneficial effects of (R)-praziquantel was shown to improve the medical compliancy because of removal of S-isomer in racemic praziquantel, a 50% component with extremely unpleasant smell and bitterness in the marketed racemic praziquantel tablet formulations. Also, it's conceivable that this removal would help to eliminate the potential concerns of safety and drug-drug (or isomer) interaction from high plasma level of the S-isomer and its hydroxyl metabolite. It is known that at the same dose level, the anthelmintic efficacy of (R)-praziquantel is better than that of praziquantel and (S)-praziquantel has almost no activity. (R)-praziquantel shows lower cardiac toxicity than (S)-praziquantel in isolated rat atria and conscious rabbits. The bitterness-free smaller pill, reduced dosage and safer profile of the current TL-010 tablet are particularly beneficial when treating odor and taste sensitive pediatric patients and pet animals such as dog and cat.

Thus, clinical investigations under both controlled protocol designs and open label trials have demonstrated that (R)-praziquantel exerts antiparasitic actions. The observed side effects after oral administration were relatively mild (drowsiness, gastric nausea or photosensitivity rash). No serious adverse reactions have been reported.

In summary, based on the anthelmintic activity, efficacy data in animals and patients with racemic praziquantel in clinical practices and preliminary reports with (R)-praziquantel by others and in this disclose, (R)-praziquantel clearly demonstrates clinical advantages when using in human subject or animals. 1) proven efficacy at lower doses; 2) better medical compliance; 3) less frequent adverse effects; and 4) fewer concerns about drug-drug or enantiomer-enantiomer interactions. The tablet formulation of the present disclosure may be administered according to certain embodiments of this disclosure to treat patients suffering from the following disorders:
1) parasitic and zoonotic diseases such as various schistosomiasis, clonorchiasis and opisthorchiasis, fasciolopsiasis, paragonimiasis, taeniases, cysticercosis, echinococcosis/hydatid disease, heterophyidiasis, echinostomiasis, neodiplostomiasis, gymnophalloidiasis, diphyllobothriasis and hymenolepiasis; etc.;
2) Parasitic infections: commonly seen in various animals include pet animal (dog, cat and horse), agriculture animals (cattle and buffalo, goat/sheep, pig and bird) as well as aquaculture and fisheries;
3) Based on the potential anti-fibrotic activity of (R)-praziquantel, the TL-010 tablet formulation of the present disclosure may be administered according to other embodiments to prevent or treat subjects suffering from hepatic fibrosis, inflammation and tissue regeneration mediated by cytokines associated with chronic schistosomiasis.

### Example 11. In Vitro Antiparasitic Activity of the (R)-Praziquantel Form B and Praziquantel Against Monogenean

(R)-praziquantel Form B was tested for use in treating fish parasites. Specifically, the effect of bath treatment of monogenean Dactylogyrus infected goldfish (Carassius auratus) was investigated.

Goldfish were acclimatized to 22° C. water (pH 7˜7.5, dissolved oxygen>5 mL/L) and were challenged with Dactylogyrus vastator. When infection rate and infection intensity reached 100% and over 10 parasite/whole gills of each fish within about 10 days, the fish were then treated with various concentrations of the (R)-praziquantel Form B or praziquantel for 48 hours by bath. Infected fish treated with the test drugs were used as a control group. 20 infected fish were placed per concentration for each treatment.

Two days after treatment, the number of attached parasites in whole gills of each fish was counted. Reduction rate=(average survival of worms in the blank control group−average survival of worms in the administration group)/average survival of worms in the blank control group×100%. The result was summarized in the table 15 below.

TABLE 15

| | Treatment | Infection | Worm |
|---|---|---|---|
| | concentration | rate | reduction |
| Group | (mg/L) | % | % |
| Blank control | 0 | 100 | |
| Praziquantel (Positive control) | 1.5 | 100 | 45 |
| (R)-praziquantel Form B | 1.5 | 100 | 98 |

The average number of adult parasites was reduced by 45% in the positive control praziquantel group and 98% in the treated group containing (R)-praziquantel Form B, respectively.

There was no toxic effect of the (R)-praziquantel Form B in the treated fish at the highest concentration of 10 mg/L.

These results clearly demonstrate that there is a therapeutic window in which (R)-praziquantel Form B can be used to effectively kill fish parasites, including Dactylogyrus vastator, but has no toxic effect on the host fish.

### Example 12. Palatability and Antiparasitic Activity of the (R)-Praziquantel Form B Against Monogenean in Yellow Croaker Infected with Neobenedenia melleni

The purpose of this experiment was to evaluate palatability and efficacy of (R)-praziquantel Form B (R-PZQ) against Monogenean trematode infection (Neobenedenia melleni) in large yellow croaker (Larimichthys crocea). Praziquantel (PZQ) was used as a positive control.

All experimental yellow croakers (about 50-75 g BW/fish) were infected with Neobenedenia melleni, and the average infection intensity was 100-120 parasites/fish.

Fish were fed 2 times a day (morning and afternoon) at up to 4% BW per day. The medicated granular feed was prepared as descripted under section “LIQUID COMPOSITIONS AND SOLID FORMULATIONS CONTAINING (R)-PRAZIQUANTEL FORM B FOR AQUACULTURE AND FISH”. Briefly, the powders of (R)-praziquantel Form B (P-PZQ) or praziquantel (PZQ) were mixed with 4% (v/v) squid oil. The resulting squid oil was subsequently top-dressed onto commercial feed granules. Control feed was prepared by top-dressing with 4% squid oil onto the feed granules without containing the compounds.

During treatment, amount and time to consume the medicated (or control) feed by the fishes in each group were observed and scored for palatability as poor (+) to excellent (+++++). Two days after dosing (Day 3), each fish was soaked in water individually and the number of parasites was recorded separately. The result was summarized in the table 16.

TABLE 16

| | | Fish | | Mortality | Infectivity |
|---|---|---|---|---|---|
| Group | Treatment | number | Palatability | % | % |
| R-PZQ | 37 mg/kg × | 30 | +++++ | 0 | 35 |
| | 2 days | | | | |
| R-PZQ | 75 mg/kg × | 30 | +++++ | 0 | 6 |
| | 2 days | | | | |
| PZQ | 75 mg/kg × | 30 | +++ | 3 | 54 |
| | 2 days | | | | |
| Control | Drug-free | 30 | ++++ | 17 | 100 |
| | feed only | | | | |

Oral treatment with R-PZQ dramatically reduced fish mortality, infectivity per fish. The antiparasitic efficacy of R-PZQ was dose dependent and superior to PZQ.

### Example 13. Acceptance Palatability Study with Soft Paste and Chew Containing (R)-Praziquantel Form B in Dogs

Following a procedure similar to that of Example 5, the palatability of the soft paste and chew compositions comprising (R)-praziquantel Form B were evaluated in dogs. Dogs in the treatment group were treated with the soft paste or chew containing (R)-praziquantel Form B described in Tables 2-4 and 10 and to provide doses of 10 mg BW/kg R-praziquantel form B. The control groups are the same soft paste and chew compositions contain praziquantel.

The prehension of the test material (Yes or No), and whether the dog consumed the soft paste or chews (fully, partially, or not at all) were recorded. At the conclusion of the study, the (R)-praziquantel soft paste and chew compositions were found have a high level of palatability (>85%) versus those containing praziquantel (40-60%).

It is to be understood that the description, specific examples and data, while indicating exemplary embodiments, are given by way of illustration and are not intended to limit the various embodiments of the present disclosure. All references cited herein for any reason, are specifically and entirely incorporated by reference. This description of the embodiments is merely to help understanding the method and the core concept of the present disclosure. It should be noted that, for those ordinary skilled in the art, various improvements and modifications can be made to the present disclosure without depart from the technical principle of the present disclosure, and these improvements and modifications also fall within the protective scope of the present disclosure.

Although the present invention has been disclosed in the form of embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

What is claimed is:

1. A pharmaceutical composition comprising a therapeutically effective amount of an anhydrous crystalline Form B of (R)-praziquantel and a pharmaceutically or veterinary acceptable carrier, the anhydrous crystalline Form B characterized by an x-ray powder diffraction pattern comprises a 2θ value of 13.1±0.2°, and the anhydrous crystalline Form B has a water content between 0 and 1% w/w assayed by standard Karl Fischer detection procedure, or the anhydrous crystalline Form B contains less than 0.2 moles of crystal water per molecular.

2. The pharmaceutical composition of claim 1, wherein the x-ray powder diffraction pattern comprises a 2θ value of 13.1±0.1°;
or the water content of the anhydrous crystalline Form B is between 0 and 0.6% w/w assayed by standard Karl Fischer detection procedure, between 0 and 0.5% w/w;
or the anhydrous crystalline Form B contains less than 0.1 moles of crystal water per molecular,
or the anhydrous crystalline Form B has no crystal water;
or the anhydrous crystalline Form B is substantially free of impurities, an impurity content is less than 1%, or the impurity content is less than 0.5%;
or the x-ray powder diffraction pattern comprises a 2θ value of 8.6±0.2° with a 100% relative intensity;
or the x-ray powder diffraction pattern comprises a 2θ value of 13.1±0.2° with a relative intensity more than 3%; and/or, the x-ray powder diffraction pattern comprises a 2θ value of 13.1±0.2° with a relative intensity lower than 7%, or lower than 5%;
or the anhydrous crystalline Form B is a white powder consisting of aggregates of birefringent rod-like or needle-like crystals;
or particle size of crystalline Form B is below 150 microns, at least 90% of the particles have a particle size of less than about 100 microns, less than about 50 microns, or less than about 20 microns, or less than about 0.2 microns;
or the purity content of the anhydrous crystalline Form B is more than 99.0%, more than 99.9%.

3. The pharmaceutical composition of claim 1, wherein the x-ray powder diffraction pattern comprises at least one 2θ value chosen from 17.9±0.2°, 19.3±0.2°, 20.1±0.2°, 20.9±0.2°, 22.3±0.2°, 24.8±0.2°, 27.8±0.2°, 31.2±0.2° and 34.5±0.2°;
or the x-ray powder diffraction pattern comprises 2θ values of 7.2±0.2°, 8.6±0.2°, 13.1±0.2°, 15.3±0.2°, 17.9±0.2°, 19.3±0.2°, 20.1±0.2°, 22.3±0.2°, 24.1±0.2°, and 24.8±0.2°;
or the x-ray powder diffraction pattern comprises 2θ values of 7.2±0.2°, 8.6±0.2°, 13.1±0.2°, 13.8±0.2°, 14.4±0.2°, 15.3±0.2°, 16.0±0.2°, 16.9±0.2°, 17.9±0.2°, 18.2±0.2°, 19.3±0.2°, 20.1±0.2°, 20.9±0.2°, 22.3±0.2°, 24.1±0.2°, 24.8±0.2°, 25.8±0.2°, 27.8±0.2°, and 31.2±0.2°;
or the x-ray powder diffraction pattern comprises 2θ values of 7.2±0.2°, 8.6±0.2°, 13.1±0.2°, 15.3±0.2°, 16.0±0.2°, 17.9±0.2°, 18.2±0.2°, 19.3±0.2°, 20.1±0.2°, 20.9±0.2°, 22.3±0.2°, 24.1±0.2°, 24.8±0.2°, 27.8±0.2°, 31.2±0.2° and 34.5±0.2°;
or the x-ray powder diffraction pattern comprises 2θ values of 7.2±0.2°, 8.6±0.2°, 13.1±0.2°, 13.8±0.2°, 14.4±0.2°, 15.3±0.2°, 16.0±0.2°, 16.9±0.2°, 17.9±0.2°, 18.2±0.2°, 19.3±0.2°, 20.1±0.2°, 20.9±0.2°, 22.3±0.2°, 24.1±0.2°, 24.8±0.2°, 25.8±0.2°, 27.8±0.2°, 31.2±0.2° and 34.5±0.2°;
and/or, the anhydrous crystalline Form B is characterized by a differential scanning calorimetry thermogram with an endotherm having a peak temperature of about 112.5° C.

4. The pharmaceutical composition of claim 1, wherein the pharmaceutical compositions are formulated as oral preparations, injection preparations, depot preparations, semi-solid preparations or topical preparations;
or the pharmaceutical compositions are formulated as tablets, capsules, pills, chews, dragees, troches, liquids, gels, pastes, solutions, syrups, slurries, granules, suspensions and the like;
or the carrier is a solid, semi-solid, or liquid material;
or the pharmaceutical composition is delivered using a sustained-release system, the sustained-release system is semi-permeable matrices of solid polymers;
or the pharmaceutical compositions take the form of tablets or chews or capsules or granules prepared with pharmaceutically acceptable excipients, and semi-solid pastes or liquid preparations prepared with pharmaceutically acceptable additives.

5. The pharmaceutical composition of claim 4, wherein the excipients comprise one or more selected from the group consisting of binders, fillers, lubricants, disintegrants, surfactants, solubilizers, glidants, flavoring agents, diluents, anti-adherents, coatings, wetting agents, or a combination thereof; the additives comprise one or more selected from the group consisting of suspending agents, emulsifying agents, solubilizers, non-aqueous vehicles, preservatives, or a combination thereof.

6. The pharmaceutical composition of claim 1, wherein the pharmaceutical composition is a tablet or a chew or a granule, including 40-80% of the anhydrous crystalline Form B by weight and 20-60% of pharmaceutically acceptable excipients by weight; the pharmaceutical composition includes 50-70% of the anhydrous crystalline Form B by weight and 30-50% of pharmaceutically acceptable excipients by weight;
and/or, the pharmaceutical composition includes 10 to 300 mg of the anhydrous crystalline Form B per tablet;
and/or, one tablet includes 10-600 mg crystalline Form B, 20-300 mg crystalline Form B, more 50-150 mg crystalline Form B.

7. The pharmaceutical composition of claim 5, wherein
the disintegrants comprise one or more selected from the group consisting of hydroxypropyl cellulose, low density hydroxypropyl cellulose, carboxymethyl starch sodium, carboxymethylcellulose, sodium carboxymethylcellulose, calcium carboxymethylcellulose, carboxymethyl starch, hydroxypropyl starch, modified starch, crystalline cellulose, sodium starch glycolate, calcium carbonate, croscarmellose sodium, crospovidone, gums, magnesium aluminum silicate, methylcellulose, polacrilin potassium, sodium alginate, low substituted hydroxypropyl cellulose, cross-linked polyvinylpyrrolidone hydroxypropyl cellulose, sodium starch glycolate, starch, or a combination thereof; and/or
the binders comprise one or more selected from the group consisting of polyvinyl pyrrolidone, povidone K30, polyethylene glycol(s), acacia, alginic acid, agar, calcium carrageenan, cellulose derivatives, ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or a combination thereof; and/or
the diluents or fillers comprise one or more selected from the group consisting of lactose, various grades of lactose, lactitol, saccharose, sorbitol, mannitol, dextrates, dextrins, dextrose, maltodextrin, croscarmellose sodium, microcrystalline cellulose, microcrystalline cellulose PH101, hydroxypropyl cellulose, L-hydroxypropyl cellulose, hydroxypropyl methylcellulose, methylcellulose polymers, hydroxyethyl cellulose, sodium carboxymethylcellulose, carboxymethylene, carboxymethyl hydroxyethyl cellulose, Non-Pareil Seeds, sugar spheres and other cellulose derivatives, starches, muddied starches, or a combination thereof; and/or
the surfactants or solubilizers comprise one or more selected from the group consisting of polysorbates, sodium dodecyl sulfate, lauryl dimethyl amine oxide, docusate sodium, poloxamers, cetyl trimethyl ammonium bromide, polyethoxylated alcohols, polyoxyethylene sorbitan, octoxynol, N, N-dimethyldodecylamine-N-oxide, hexadecyltrimethylammonium bromide, polyoxyl 10 lauryl ether, brij, bile salts, polyoxyl castor oil, 2-hydroxylpropyl-beta-cyclodextrin, β-cyclodextrin, sulfobutylether β-cyclodextrin, RMβCD, lecithin, methylbenzethonium chloride, carboxylates, sulphonates, petroleum sulphonates, alkylbenzenesulphonates, naphthalenesulphonates, olefin sui phonates, alkyl sulphates, sulphates, sulphated natural oils & fats, sulphated esters, sulphated alkanolamides, alkylphenols, ethoxylated & sulphated, ethoxylated aliphatic alcohol, polyoxyethylene surfactants, carboxylic esters polyethylene glycol esters, anhydrosorbitol ester & it's ethoxylated derivatives, glycol esters of fatty acids, carboxylic amides, monoalkanolamine condensates, polyoxyethylene fatty acid amides, quaternary ammonium salts, amines with amide linkages, polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt, or a combination thereof; and/or
the glidants, anti-adherents and lubricants comprise one or more selected from the group consisting of stearic acid and pharmaceutically acceptable salts or esters, talc, waxes, glycerides, light mineral oil, PEG, silica acid or a derivative or salt thereof, sucrose ester of fatty acids, hydrogenated vegetable oils or a combination thereof; and/or
the disintegrants are present in an amount ranging from 2% to 30% by weight, or 2% to 10% by weight, or 2% to 8% by weight; and/or, the disintegrant is sodium starch glycolate;
and/or, the binders are present in an amount ranging from 1% to 20% by weight, or 2% to 8% by weight; and/or, the binder is povidone K30;
and/or, the carriers, diluents or fillers are present in an amount ranging from 2% to 50% by weight, or 17.5% to 45% by weight, or 35% to 45% by weight; and/or, the diluent or filler is microcrystalline cellulose;
and/or, the surfactants or solubilizers are present in an amount ranging from 0.5% to 10% by weight, 1% to 3% by weight, 0.9% to 1% by weight; and/or, the surfactant or solubilizer is sodium lauryl sulfate;
and/or, the glidants, anti-adherents or lubricants are present in an amount ranging from 0.1% to 6% by weight; or 0.1% to 5% by weight; and/or, the glidant is colloidal silicon dioxide, and its content is 0.3-2%; and/or, the lubricant is sodium stearyl fumarate, and its content is 0.3-5%, or 0.3-3%; or the lubricant is magnesium stearate, and its content is 0.25-5%.

8. The pharmaceutical composition of claim 1, wherein the pharmaceutical formulation is a tablet, including 40-80% of crystalline Form B by weight, 30˜50% of microcrystalline cellulose by weight, 0.5-2% of sodium lauryl sulfate by weight, 2-8% of sodium starch glycolate by weight, 1-8% of povidone K30 by weight, 0.3-2% of colloidal silicon dioxide by weight, 0.1-5% of sodium stearyl fumarate by weight.

9. The pharmaceutical composition of claim 8, wherein the tablet includes 40-60% of crystalline Form B by weight, 30-50% of microcrystalline cellulose by weight, 0.5-2% of sodium lauryl sulfate by weight, 5-8% of sodium starch glycolate by weight, 1-5% of povidone K30 by weight, 0.3-1% of colloidal silicon dioxide by weight, 0.3-3% of sodium stearyl fumarate by weight.

10. The pharmaceutical composition of claim 1, wherein the pharmaceutical composition is a semi-solid oral homogeneous or granular or chewable anthelmintic veterinary formulation, for the treating, controlling, and preventing of endo- and ectoparasite infections in animals, the semi-solid oral homogeneous anthelmintic veterinary formulation comprises the anhydrous crystalline Form B alone or combined with, at least one additional veterinary agent selected from abamectin, Selamectin, moxidectin, ivermectin, emamectin, doramectin, eprinomectin, pyrantel, amitraz, albendazole, cambendazole, fenbendazole, flubendazole, mebendazole, febantel, octadepsipeptides, Oxfendazole, oxibendazole, paraherquamide, parbendazole, thiabendazole, tetramisole, triclabendazole, levamisole, oxantel, novaluron, morantel, milbemycin, milbemycin oxime, cyclo-octadepsipeptides, demiditraz, diethylcarbamazine, fipronil, hydroprene, kinoprene, methoprene, metaflumizone, niclosamide, permethrin, pyrethrins, pyriproxyfen, spinosad, aminoacetonitrile derivative(s), pyrantel, benzimidazoles, tribendimidine, or any mixture thereof, and veterinary acceptable inert ingredients;
the combination therapy includes following active pharmaceutical ingredients:
1.5% (R)-praziquantel form B or plus 0.15% moxidectin or plus 0.25% milbemycin oxime or plus 25% tribendimidine or plus albendazole;
and/or, the pharmaceutical composition is a semi-solid paste or a chew, including 1-90% of the anhydrous crystalline Form B by weight and over 10% of pharmaceutically acceptable excipients by weight;
and/or, the pharmaceutical composition is a granule, including 1-90% of the anhydrous crystalline Form B by weight and over 10% of pharmaceutically acceptable excipients by weight.

11. The pharmaceutical composition of claim 10, wherein the veterinary acceptable inert ingredients comprise one or more selected from the group consisting of fillers, thickeners, flavoring agents, binders, colorants or opacifiers, humectants, preservatives, antioxidants, cyclodextrin, buffer, viscosity modifiers, solubilizers, surfactants, lubricants, antimicrobial agents, solvents or a combination thereof;
the fillers are present in an amount ranging from 1% to 40% by weight, and/or, the filler is one or more selected from the group consisting of corn starch, soy protein fines, microcrystalline cellulose, or a combination thereof;
and/or, the thickeners are present in an amount ranging from 1% to 30% by weight, and/or, the thickener is one or more selected from the group consisting of hydroxypropyl cellulose, microcrystalline cellulose, corn starch, xanthan gum, polyvinylpyrrolidone, hydroxyethyl starch or a combination thereof;
and/or, the flavoring agents are present in an amount ranging from 10% to 25% by weight;
and/or, the binders are present in an amount ranging from 2% to 8% by weight; and/or, the binder is one or more selected from the group consisting of polyvinylpyrrolidone K30, cross-linked polyvinylpyrrolidone, PEG 4000, or a combination thereof;
and/or, the colorants or opacifiers are present in an amount ranging from 0% to 2% by weight;
and/or, the humectants are present in an amount ranging from 1% to 25% by weight; and/or, the humectant is one or more selected from the group consisting of glycerol, animal fats, fish oil, propylene glycol, or a combination thereof;
and/or, the preservatives are present in an amount ranging from 0.3% to 1.4% by weight; and/or, the preservative is one or more selected from the group consisting of benzyl alcohol, sodium benzoate, potassium sorbate, or a combination thereof;
and/or, the antioxidants are present in an amount ranging from 0.01% to 7% by weight; and/or, the antioxidant is one or more selected from the group consisting of butylated hydroxy anisole, butylated hydroxytoluene, ascorbic acid, or a combination thereof;
and/or, the cyclodextrin are present in an amount ranging from 0.5% to 40% by weight;
and/or, the buffer is HCL or citrate;
and/or, the viscosity modifiers are present in an amount ranging from 1% to 10% by weight; and/or, the viscosity modifier is one or more selected from the group consisting of hydrogenated castor oil, corn oil, olive oil, or a combination thereof;
and/or, the solubilizers are present in an amount ranging from 1% to 5% by weight; and/or, the solubilizer is HPβCD;
and/or, the surfactants are present in an amount ranging from 1% to 5% by weight; and/or, the surfactant is sodium lauryl sulfate;
and/or, the solvents are present in an amount ranging from 1% to 10% by weight; and/or, the solvent is propylene glycol, polyethylene glycol or glycerol;
and/or, the lubricants are present in an amount ranging from 1% to 10% by weight; and/or, the lubricant is hydrogenated vegetable oils;
and/or, the antimicrobial agents are present in an amount ranging from 1% to 10% by weight; and/or, the antimicrobial agent is benzyl alcohol;
and/or, the semi-solid oral homogeneous is an oil based paste, the veterinary acceptable inert ingredients comprise one or more selected from the group consisting of fillers, surfactants, flavoring agents, thickeners, binders, solvents, lubricants, preservatives, antimicrobial agents, antioxidants, opacifiers, or a combination thereof;
and/or, the semi-solid oral homogeneous is an aqueous based paste, the veterinary acceptable inert ingredients comprise one or more selected from the group consisting of fillers, colorants or opacifiers, solubilizers, flavoring agents, binders, humectants, preservatives, buffers, or a combination thereof; and
the buffer is citrate.

12. The pharmaceutical composition of claim 1, wherein the pharmaceutical composition further comprises one or more selected from the group consisting of tinidazole, metronidazole, melarsoprol, eflornithine, rifampin, amphotericin B, pentamidine, sodium stibogluconate, meglumine antimoniate, fluconazole, artesunate, artemether, dihydroartemisinin, quinine, quinidine, chloroquine, atovaquone-proguanil, artemether-lumefantrine, mefloquine, doxycycline, clindamycin, paromomycin, atovaquone, nitazoxanide, azithromycin, fumagillin, paromomycin, diloxanide, secnidazole, ornidazole, iodoquinol, diloxanide furoate, clindamycin, atovaquone, azithromycin, diminazen, trypan blue, oxamniquinine, emodepside, tribendimidine, fenbentel, monepantel, derquantel, diethylcarbamazine, abamectin, selamectin, moxidectin, ivermectin, emamectin, doramectin, eprinomectin, pyrantel, amitraz, albendazole, cambendazole, fenbendazole, flubendazole, mebendazole, febantel, octadepsipeptides, Oxfendazole, oxibendazole, paraherquamide, parbendazole, thiabendazole, tetramisole, triclabendazole, levamisole, oxantel, novaluron, morantel, milbemycin, milbemycin oxime, demiditraz, diethylcarbamazine, fipronil, hydroprene, kinoprene, methoprene, metaflumizone, niclosamide, permethrin, pyrethrins, pyrantel pamoate, pyriproxyfen, spinosad and aminoacetonitrile derivative.

13. The pharmaceutical composition of claim 1, wherein the anhydrous crystalline Form B is prepared by the following steps:
a) dissolving the compound of (R)-praziquantel in at least one solvent at around 45° C. to form a solution;
b) adding another solvent to the solution and cooling down to form a solid;
c) isolating the solid by filtration;
d) drying the solids under vacuum with a pressure≤−0.08 MPa at 35±5° C. for at least 10 hours to get the anhydrous crystalline Form B.

14. The pharmaceutical composition of claim 13, wherein the one solvent is anhydrous solvent, the one solvent is one or more selected from the group consisting of acetone, acetonitrile, chloroform, dichloromethane, dimethyl sulfoxide, 1,2-dimethoxyethane, 1,4-dioxane, ethanol, ethyl acetate, isopropyl acetate, isopropyl alcohol, methanol, methyl isobutyl ketone, 2-methyltetraohydrofuran, methyl tert-butyl ether, N,N-dimethylacetamide, N-methyl pyrrolidone, tetrahydrofuran, toluene; and the another solvent is one or more selected from the group consisting of n-heptane, n-hexane and petroleum ether;
and/or, in step b), cooling down the mixture at 0˜5° C. for at least 5 hours.

15. A method for preventing or treating subjects suffering from parasitic infections, diseases or associated disorders in a subject, the method comprising administering the pharmaceutical composition according to claim 1 to the subject suffering from the parasitic disease.

16. The method of claim 15, wherein the parasitic infections, diseases and associated disorders with cestodes and trematodes, include but is not limited to, schistosomiasis, clonorchiasis, paragonimiasis, opisthorchiasis, fasciolopsiasis, taeniasis (tapeworm infection), cysticercosis, echinococcosis, heterophyidiasis, echinostomiasis, neodiplostomiasis, gymnophalloidiasis, diphyllobothriasis, bertielliasis, sparganosis and hymenolepiasis;
and/or inflammation, liver fibrosis and regeneration mediated by worm-specific cytokine in the course of the infections.

17. The method of claim 15, wherein a dosage ranges from 0.1 to 4500 mg per day, and/or
the method further comprises administering the pharmaceutical composition to the subject one or more times a day, with a total daily intake of crystalline Form B greater than 10 mg/kg for a human or greater than 1 mg/kg for an animal; and/or administered daily for consecutive days.

18. The method of claim 15, wherein the subject is human, animal or fish; and/or the subject is cats, dog, horse, buffalo, cattle, pig, sheep, goat or fish or bird.

19. A method for treating with the pharmaceutical composition according to claim 1 in aquaculture animals and fishes for the infections caused by parasites.

20. The method of claim 19, wherein
the parasites are cestodes and trematodes in various fishes and reptiles; and/or
the parasites include but is not limited to: 1) Monogeneans: Ancylodiscoides vistulensis, Benedenia seriolae, Neobenedenia, Cleidodiscus sp., Clemacotyle australis, Dactylogyrus sp., Gyrodactylus aculeati, Gyrodactylus turnbulli, Haliotrema abaddon, Lepidotrema bidyanain in European catfish, Yellowtail amberjack, Black crappie, Whitespotted eagle rays, goldfish, guppy, stickleback, West Australian dhufish, silver perch, golden pomfrets, yellow croaker, eels and 2) Digeneans: Clinostomum complanatum, Clinostomum marginatum, Diplostomum spathaceum in sunshine bass, channel catfish, grass carp, silver carp, and 3) Cestodes: Bothriocephalus acheilognathi in Bonytail chub, grass carp, red shiner, red snapper, as well as aporocotylid blood flukes in bluefin tuna, Cardicola forsteri, Thunnus maccoyii, Thunnus thynnus, Thunnus orientalis and Cardicola orientalis; and/or
the administration method includes medicinal bath, medicated feed or oral intubation; and/or
for medicinal bath, a final concentration of the anhydrous crystalline Form B in water is from 1 mg/L to 50 mg/L for up to 96 hours, preferable at 10 mg/L between 1 hour to 48 hours, more preferable at 5 mg/L between 6 hours to 24 hours; and/or
for oral administration, a formulation comprising (R)-praziquantel Form B in the form of fish feed granules or pellets or pills or powders with feed additives, and/or
an amount of (R)-praziquantel Form B ranges from is 5 mg/kg to 200 mg/kg body weight, single, or twice a day or daily doses up to 20 days, preferable at 100 mg/kg body weight, single dose, more preferable between 30 to 100 mg/kg single or twice a day for 2 to 5 consecutive days.
